SEQUENCE LISTING (17 sequences)

SEQ ID NO: 1 - protein, 74 aa, Bacillus stearothermophilus
MYTNSDFVVIKALEDGVNVIGLTRGADTRFHHSEKLDKGEVLIAQFTEHTSAIKVRGKAYIQTRHGVIESEGKK

SEQ ID NO: 2 - DNA, 225 bp, Bacillus stearothermophilus
atgtatacgaacagcgactttgttgtcattaaagcgcttgaagacggagtgaacgtcattggattgacgcgcggggcggatacacggttccatcactcggaaaagctcgataaaggcgaagtgttgatcgcccagtttacagagcacacgtcggcgattaaagtgagaggcaaggcgtatattcaaacgcgccatggcgtcattgagtcggaagggaaaaagtaa

SEQ ID NO: 3 - protein, 74 aa, artificial
  TRAP-K35C-E48Q
MYTNSDFVVIKALEDGVNVIGLTRGADTRFHHSECLDKGEVLIAQFTQHTSAIKVRGKAYIQTRHGVIESEGKK

SEQ ID NO: 4 - protein, 74 aa, artificial
  TRAP-K35C-E48K
MYTNSDFVVIKALEDGVNVIGLTRGADTRFHHSECLDKGEVLIAQFTKHTSAIKVRGKAYIQTRHGVIESEGKK

SEQ ID NO: 5 - protein, 74 aa, artificial
  TRAP-K35C
MYTNSDFVVIKALEDGVNVIGLTRGADTRFHHSECLDKGEVLIAQFTEHTSAIKVRGKAYIQTRHGVIESEGKK

SEQ ID NO: 6 - protein, 74 aa, artificial
  TRAP-K35C R64S
MYTNSDFVVIKALEDGVNVIGLTRGADTRFHHSECLDKGEVLIAQFTEHTSAIKVRGKAYIQTSHGVIESEGKK

SEQ ID NO: 7 - protein, 249 aa, artificial
  GFP(-21)
  misc_feature (16)..(16): Xaa can be any naturally occurring amino acid
  misc_feature (19)..(20): Xaa can be any naturally occurring amino acid
HHHHGSACELMVSKGXELXXGVVPILVELDGDVNGHEFSVRGEGEGDATEGELTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHDVYITADKQENGIKAEFEIRHNVEDGSVQLADHYQQNTPIGDGPVLLPDDHYLSTESALSKDPNEKRDHMVLLEFVTAAGITHGMDELYK

SEQ ID NO: 8 - protein, 12 aa, artificial
  PTD4 derivative
  MISC_FEATURE (1)..(1): Acetylation of the N-terminal amino group
YARAAARQARAG

SEQ ID NO: 9 - protein, 11 aa, artificial
  PTD4
YARAAARQARA

SEQ ID NO: 10 - protein, 335 aa, artificial
  His6-mCherry-TRAP-K35C
MHHHHHHGGSSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKLSENLYFQSGGSGSSYTNSDFVVIKALEDGVNVIGLTRGADTRFHHSECLDKGEVLIAQFTEHTSAIKVRGKAYIQTRHGVIESEGKK

SEQ ID NO: 11 - protein, 335 aa, artificial
  His6-mOrange-TRAP-K35C
MHHHHHHGGSSMVSKGEENNMAIIKEFMRFKVRMEGSVNGHEFEIEGEGEGRPYEGFQTAKLKVTKGGPLPFAWDILSPHFTYGSKAYVKHPADIPDYFKLSFPEGFKWERVMNYEDGGVVTVTQDSSLQDGEFIYKVKLRGTNFPSDGPVMQKKTMGWEASSERMYPEDGALKGKIKMRLKLKDGGHYTSEVKTTYKAKKPVQLPGAYIVDIKLDITSHNEDYTIVEQYERAEGRHSTGGMDELYKLSENLYFQSGGSGSSYTNSDFVVIKALEDGVNVIGLTRGADTRFHHSECLDKGEVLIAQFTEHTSAIKVRGKAYIQTRHGVIESEGKK

SEQ ID NO: 12 - protein, 300 aa, artificial
  His6-SUMO-SpyCatcher-TRAP-K35C
MHHHHHHGSSMASMKDHLIHNHHKHEHAHAEHLGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSDSATHIKFSKRDEDGKELAGATMELRDSSGKTISTWISDGQVKDFYLYPGKYTFVETAAPDGYEVATAITFTVNEQGQVTVNGKATKGDAHIGSSYTNSDFVVIKALEDGVNVIGLTRGADTRFHHSECLDKGEVLIAQFTEHTSAIKVRGKAYIQTRHGVIESEGKK

SEQ ID NO: 13 - protein, 309 aa, artificial
  His6-SUMO-TRAP-K35C
MHHHHHHGSSMASMKDHLIHNHHKHEHAHAEHLGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSGSGGSSYTNSDFVVIKALEDGVNVIGLTRGADTRFHHSECLDKGEVLIAQFTGSSDSATHIKFSKRDEDGKELAGATMELRDSSGKTISTWISDGQVKDFYLYPGKYTFVETAAPDGYEVATAITFTVNEQGQVTVNGKATKGDAHIPGTEHTSAIKVRGKAYIQTRHGVIESEGKK

SEQ ID NO: 14 - protein, 117 aa, artificial
  SpyTag-TRAP-K35C-srt-His6
MAHIVMVDAYKPTKQGSGGSGSSYTNSDFVVIKALEDGVNVIGLTRGADTRFHHSECLDKGEVLIAQFTEHTSAIKVRGKAYIQTRHGVIESEGKKGTGGSLPSTGGAPVEHHHHHH

SEQ ID NO: 15 - protein, 268 aa, artificial
  His6-SpyTag-GFP
MGSSHHHHHHGGSAHIVMVDAYKPTKGSGTASKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYK

SEQ ID NO: 16 - protein, 130 aa, artificial
  His6-SpyTag-NL-2
MGSSHHHHHHGGSAHIVMVDAYKPTKGSGTPKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 17 - protein, 72 aa, artificial
  TRAP mutant K35C, R64S, dK73, dK74K
MYTNSDFVVIKALEDGVNVIGLTRGADTRFHHSECLDKGEVLIAQFTEHTSAIKVRGKAYIQTSHGVIESEG

(19) United States
(12) Patent Application Publication
(10) Pub. No.: US 2024/0181077 A1
(43) Pub. Date: Jun. 6, 2024

(54) AN ARTIFICIAL PROTEIN-CAGE COMPRISING ENCAPSULATED THEREIN A GUEST CARGO

(71) Applicant: UNIWERSYTET JAGIELLONSKI, Krakow (PL)

(72) Inventors: Jonathan HEDDLE, Krakow (PL); Artur BIELA, Krakow (PL); Yusuke AZUMA, Krakow (PL); Antonina NASKALSKA, Krakow (PL); Kinga BORZECKA-SOLARZ, Krakow (PL); Jan ROZYCKI, Krakow (PL); Izabela STUPKA, Krakow (PL)

(21) Appl. No.: 18/547,242

(22) PCT Filed: Feb. 24, 2022
(86) PCT No.: PCT/PL2022/050011

§ 371 Date: Oct. 10, 2023

(30) Foreign Application Priority Data
Feb. 24, 2021 (LU) LU102569
Feb. 24, 2021 (LU) LU102571
Feb. 24, 2021 (LU) LU102572
Feb. 24, 2021 (PL) P.437113
Feb. 24, 2021 (PL) P.437114
Feb. 24, 2021 (PL) P.437115

(51) Int. Cl. A61K 47/69; A61K 39/385; A61K 41/00

(52) U.S. Cl. CPC A61K 47/6949; A61K 39/385; A61K 41/0042; A61K 2039/6031

(57) ABSTRACT

The present invention provides an artificial TRAP-cage comprising a selected number of TRAP rings and encapsulated therein a guest cargo.

### FIELD OF THE INVENTION

The present invention falls within the biochemistry field. It is related to an artificial protein cage called “TRAP-cage” comprising a selected number of TRAP rings and encapsulated therein a guest cargo.

### BACKGROUND

Proteins that assemble into monodisperse cage-like structures are useful molecular containers for diverse applications in biotechnology and medicine. Such protein cages exist in nature, e.g. viral capsids, but can also be designed and constructed in the laboratory.

As such, inventors previously described that a single cysteine mutant of the tryptophan RNA-binding attenuation protein from Geobacillus stearothermophilus, TRAP-K35C, can assemble into a hollow spherical structure composed of multiple ring-shape undecameric subunits via reaction with gold nanoparticles¹.The resulted protein cages show an extremely high stability under many harsh conditions, but easily disassemble to the capsomer units by addition of reducing agents.

Although those appealing characteristics of the TRAP cages are ideal to develop an intracellular delivery vehicle, an essential challenge has remained guest packaging.

The object of the invention is to provide a facile and robust method for internal loading of the TRAP-cages with proteins or therapeutics of interest in a stoichiometry controllable manner.

### SUMMARY OF THE INVENTION

The subject matter of the invention is an artificial TRAP-cage comprising a selected number of TRAP rings and encapsulated therein at least one guest cargo. Preferably, the artificial TRAP-cage comprises a selected number of TRAP rings which are held in place by cross-linkers. Preferably, the cross-linkers are molecular cross linkers or atomic metal cross linkers. Preferably the TRAP rings are linked by gold or DTME.

Preferably, the guest cargo is no larger than the diameter of the TRAP-cage lumen. Preferably the guest cargoes are below 16 nm in diameter. Preferably the guest cargoes are between 4 nm and 16 nm in diameter. Guest cargoes larger than 4 nm would be unable to diffuse into, or out of the TRAP-cages.

Preferably, the cargo is a protein, preferably selected from the group comprising an enzyme (e.g. protease, a nuclease, hydrogenase, dehydrogenase, lipase, lyase, ligase, transferase, reductase, recombinase, nuclease acid modification enzyme. or other type of enzyme) an antigen, an antibody. Or the cargo is another type of protein biological macromolecule (e.g. a sterol, steroid or a fatty acid). Or the cargo is a lipid, a peptide (e.g. a peptide hormone, a cell membrane disrupting peptide, a T-cell-stimulating peptide or another type of peptides) a nucleic acid (e.g. DNA, designed DNA nanostructures including those designed using the DNA origami technique, DNAzymes, RNA, mRNA, miRNA, siRNA, tRNA single stranded RNA, double stranded RNA, RNAzymes), a small molecular cargo such as a drug, a peptide nucleic acids (PNA), a carbon-based structure (e.g. a fullerene or a buckminsterfullerene, a single walled carbon nanotube or a multi-walled carbon nanotube) a metal (e.g. iron, zinc, platinum, copper, sodium, cadmium, lanthanides, gadolinium, technetium, calcium, potassium, chromium, magnesium, molybdenum and salts or complexes thereof), a toxin (e.g. a ligand targeted toxin, a protease activated toxin, melittin and a toxin-based suicide gene therapeutic) or a nanoparticle (e.g. a metal nanoparticle such as gold, iron, silver, cobalt cadmium selenide, titanium oxide) or a core-shell metal nanoparticle such as CdS/ZnS, CdSe/ZnS, CdSeICdS, and InAs/CdSe nanoparticle.

Preferably, the nucleic acid is selected from the group comprising DNA, RNA, mRNA, siRNA, tRNA and micro-RNA.

Preferably, the therapeutic agent is an enzyme associated with an over-expression in a metabolic disorder or disease or an under expression in a metabolic disorder or disease.

Preferably, the enzyme is selected from the group comprising hydrogenase, dehydrogenase, lipase, lyase, ligase, protease, transferase, reductase, recombinase and nuclease acid modification enzyme.

Preferably, the therapeutic agent is selected from the group comprising a cancer therapeutic, an anti-infection therapeutic, a vascular disease therapeutic, an immune therapeutic, senolytic and a neurological therapeutic.

Preferably, the metal is selected from the group comprising iron, zinc, platinum, copper, sodium, cadmium, lanthanide, gadolinium, technetium, calcium, potassium, chromium, magnesium, molybdenum and salts or complexes thereof.

Preferably, the toxin is selected from the group comprising a ligand targeted toxin, a protease activated toxin, melittin and a toxin-based suicide gene therapeutic.

Preferably, the guest cargo is a protein. Preferably a fluorescent protein. Preferably GFP, mCherry or mOrange. Preferably interleukin-2 (IL-2) or Neoleukin-2/15 (NL-2).

Preferably, the therapeutic agent is selected from the group comprising a cancer therapeutic, an anti-infection therapeutic, a vascular disease therapeutic, an immune therapeutic, senolytic and a neurological therapeutic.

Preferably, the cage comprises multiple cargoes, preferably the cargoes are the same or different from one another.

Preferably, the TRAP-cage according to the invention further includes at least one external decoration.

Preferably, at least one of the external decorations comprises a cell penetrating agent to promote intracellular delivery of the cage containing an internal guest cargo.

Preferably, the cell penetrating agent is PTD4.

Preferably, wherein the number of TRAP rings in the TRAP-cage is between 6 and 60, preferably between 7 and 55, preferably between 8 and 50, preferably between 9 and 45, preferably between 10 and 40, preferably between 11 and 35, preferably between 12 and 34, preferably between 13 and 33, preferably between 14 and 32, preferably between 15 and 31, preferably between 16 and 30, preferably between 17 and 29, preferably between 18 and 28, preferably between 19 and 27, preferably between 20 and 26. Preferably the number of TRAP rings in the TRAP-cage is less than 40, preferably less than 35, preferably less than 30. Preferably the number of TRAP rings in the TRAP-cage is more than 6, preferably more than 10, preferably more than 15, preferably more than 20.

Preferably, the number of TRAP rings in the TRAP-cage is between 12 and 24.

Preferably, the number of TRAP rings in the TRAP-cage is about 24, preferably 24. Preferably, the number of TRAP rings in the TRAP-cage is about 12, preferably 12. Preferably, the number of TRAP rings in the TRAP-cage is about 20, preferably 20.

Preferably, the interior surface of the TRAP-cage lumen is supercharged. Preferably the TRAP-cage with a supercharged lumen comprises a E48Q or a E48K mutation. Preferably the TRAP-cage with a supercharged lumen comprises a K35C/E48Q or a K35C/E48K mutation.

Preferably, the guest cargo is genetically fused to the interior surface of the TRAP-cage lumen. Preferably, the genetic fusion of the guest cargo to an interior surface of the TRAP cage lumen of step (ii) is via N-terminus fusion of the guest cargo to an N-terminus of TRAP^{K35C} which faces into the interior surface of the lumen. Preferably the guest cargo is a genetically fused protein, preferably a fluorescent protein, preferably GFP, mCherry or mOrange. Preferably the genetically fused protein is interleukin-2 (IL-2) or Neoleukin-2/15 (NL-2).

Preferably, the guest cargo is conjugated using SpyCatcher/SpyTag conjugation, preferably to an interior surface of the TRAP-cage lumen. Preferably, the SpyCatcher/SpyTag conjugation of the guest cargo to an interior surface of the TRAP-cage lumen of step (iii) wherein the SpyCatcher is introduced in a loop region of TRAP rings between residues 47 and 48, which faces to the interior when assembled into TRAP-cages and the guest cargo contains a SpyTag.

Preferably, the guest cargo is attached via a covalent bond to the TRAP cage, preferably inside the TRAP Cage, preferably by chemical or enzymatic bond formation.

Preferably, opening of the cage is programmable. Preferably, said specific conditions corresponds to the specific cleavage characteristic of the cross-linker.

Preferably, the programmable opening of the cage is dependent on selection of a molecular or atomic metallic cross-linkers which hold the TRAP-rings in place in the TRAP-cage.

Preferably, the specific cleavage characteristic of the molecular cross-linker is selected from the group comprising:
(i) a reduction resistant/insensitive molecular cross-linker, whereby the cage remains closed under reducing conditions;
(ii) a reduction responsive/sensitive molecular cross-linker, whereby the cage opens under reducing conditions; and
(iii) a photoactivatable molecular cross-linker whereby the cage opens upon exposure to light.

Preferably, the reduction resistant/insensitive molecular cross-linker can be selected from the group comprising: bismaleimideohexane (BMH) and bis-bromoxylenes. Preferably, the reduction responsive/sensitive molecular cross-linker can be selected from the group comprising: dithiobismaleimideoethane (DTME). Preferably, the photoactivatable molecular cross-linker can be selected from the group comprising: bis-halomethyl benzene and its derivatives including 1,2-bis-bromomethyl-3-nitrobenzene (o-BBN), 2,4-bis-bromomethyl-1-nitrobenzene (m-BBN) and 1,3-bis-bromomethyl-4,6-dinitro-benzene (BDNB).

Preferably, the molecular cross-linker is a homobisfunctional molecular moiety and its derivatives. Preferably, homobisfunctional molecular cross-linker is bismaleimideohexane (BMH).

Preferably, the cage is resistant/insensitive to reducing conditions. Preferably the homobisfunctional molecular cross-linker is dithiobismaleimideoethane (DIME).

Preferably, the cage is responsive/sensitive to reducing conditions. Preferably the molecular cross-linker is a bis-halomethyl benzene and its derivatives.

Preferably, the molecular cross-linker is selected from the group comprising, 1, 2-bis-bromomethyl-3-nitrobenzene (BBN), bis-bromoxylene and 1,3-bis-bromomethyl-4,6-dinitro-benzene (BDNB).

Preferably, the molecular cross-linker is photolabile by exposure to UV light.

Preferably, the cage according to the invention comprises a mixture of different programmable molecular cross-linkers.

Preferably, the TRAP rings are variants.

Preferably, the artificial TRAP-cage protein is modified to comprise any one or more of the following mutations selected from the group comprising K35C, E48Q, E48K R64S, K35C/E48Q, K35C/E48K, and K35C/R64S. Preferably the artificial TRAP-cage protein is modified to comprise a K35C mutation. Preferably the artificial TRAP-cage protein is modified to comprise a K35C mutation or a K35C/E48Q mutation or a K35C/E48K mutation.

Preferably, the artificial TRAP-cage protein is modified to comprise any one or more of the following mutations selected from the group comprising K35C, K35H, R64S, E48Q, E48K, K35C/R64S, K35H/R64S, S33C, S33H, S33C/R64S, S33H/R64S, S33C/K35H S33H/K35H, S33C/K35C, S33H/K35C, K35C/E48Q, K35C/E48K, K35H/E48Q, K35H/E48K, S33C/E48Q, S33C/E48K, S33C/E48Q, S33C/R64S/E48Q, K35H/S33C/R64S, S33C/R64S, S33H/K35H/R64S, S33C/K35C/R64S, K35C/R64S/E48Q, K35H/R64S/E48Q, K35H/R64S/E48K, S33C/R64S/E48Q, S33C/R64S/E48K, S33C/R64S/E48Q and S33C/E48K. Preferably the artificial TRAP-cage protein is modified to comprise a K35H/E48Q or a K35H/E48 mutation.

Preferably, the TRAP-cages are stable in elevated temperatures, i.e. when the temperatures are elevated above normal room or human/animal body temperatures, preferably stable between 0 and 100° C., preferably stable between 15 and 100° C., preferably stable between 15 and 79° C., preferably stable up to 95° C., preferably stable at 95° C. and below.

Preferably, the TRAP-cages are stable in a non-neutral pH, preferably stable above pH 7 and below pH 7, preferably stable between pH 3 to 11, preferably stable between pH 4 to 10, preferably stable between pH 5 to 9.

Preferably, the TRAP-cages are stable in chaotropic agents (agents which disrupt hydrogen bonding in solution, which would disrupt or denature protein or macromolecular structures) or surfactants that would otherwise be expected to disrupt or denature protein or macromolecular structures. Preferably the cages show stability in n-butanol, ethanol, guanidinium chloride, lithium perchlorate, lithium acetate, magnesium chloride, phenol, 2-propanol, sodium dodecyl sulfate, thiourea, and urea. Preferably, the TRAP-cages are stable in up to 4 M GndHCI. Preferably, the TRAP-cages are stable in up to at least 7 M urea. Preferably, the TRAP-cages are stable in up to 15% of SDS. The stability of the cages described herein can be tested in standard conditions which would be known to the person of skill in the art using these agents to demonstrate said stability.

The cages described herein display unexpected stability in these conditions, providing more stable TRAP-cages than previously demonstrated.

The subject matter of the invention is also use of the cage according to the invention, as defined above, in delivery of a cargo in a controlled period and to a desired location.

The subject matter of the invention is also the use of the artificial TRAP-cage according to the invention as a delivery vehicle for intracellular delivery of its internal guest cargo.

The subject matter of the invention is also the use of the artificial TRAP-cage according to the invention as a vaccine.

The subject matter of the invention is also use of the artificial TRAP-cage according to the invention for the treatment of an illness or disease condition selected from the group comprising cancer, vascular disease, cardiovascular disease, diabetes, infection, auto-immune condition, neurodegenerative disease, cellular senescence disease, arthritis and respiratory disease.

The subject matter of the invention is also a method of making an artificial TRAP-cage with an encapsulated guest cargo, the method comprising:
(i) obtaining TRAP ring units by expression of the TRAP ring units in a suitable expression system and purification of the said units from the expression system;
(ii) conjugation of the TRAP ring units via at least one free thiol linkage with a cross-linker;
(iii) modification of the TRAP ring units to provide a suitable interior surface environment for capturing a guest cargo;
(iv) formation of the TRAP-cage by self-assembly to provide a cage lumen wherein the guest cargo is encapsulated; and
(v) purification and isolation of the TRAP-cages encapsulating the guest cargo.

Preferably, step (ii) first comprises conjugation of the TRAP ring units via at least one metal cross-linker, preferably an atomic metal cross-linker. Step (ii) then comprises replacing the metal cross-linker with a molecular cross-linker. A molecular cross-linker may exchange metal atoms without changing orientation of the rings in the cage. Preferably, the metal is gold. This altered step (ii) preferably applies when the cross-linker is a photocleavable linkers, preferably wherein the cross linker is bromoxylene or bisbromobimane.

Preferably the modification of step (iii) is selected from the group comprising:
(i) super charging the interior surface of the TRAP-cage lumen;
(ii) genetic fusion of the guest cargo to an interior surface of the TRAP-cage lumen;
(iii) SpyCatcher/SpyTag conjugation of the guest cargo to an interior surface of the TRAP-cage lumen; and
(iv) via covalent bond formation in both chemical and enzymatic methods.

Preferably, the super charging of step (i) of the interior surface provides either a net positive or net negative charge on the interior surface of the cage lumen.

Preferably, the cargo is a protein, preferably selected from the group comprising an enzyme (e.g. protease, a nuclease, hydrogenase, dehydrogenase, lipase, lyase, ligase, transferase, reductase, recombinase, nuclease acid modification enzyme. or other type of enzyme) an antigen, an antibody. Or the cargo is another type of protein biological macromolecule (e.g. a sterol, steroid or a fatty acid). Or the cargo is a lipid, a peptide (e.g. a peptide hormone, a cell membrane disrupting peptide, a T-cell-stimulating peptide or another type of peptides) a nucleic acid (e.g. DNA, designed DNA nanostructures including those designed using the DNA origami technique, DNAzymes, RNA, mRNA, miRNA, siRNA, tRNA single stranded RNA, double stranded RNA, RNAzymes), a small molecular cargo such as a drug, a peptide nucleic acids (PNA), a carbon- based structure (e.g. a fullerene or a buckminsterfullerene, a single walled carbon nanotube or a multi-walled carbon nanotube) a metal (e.g. iron, zinc, platinum, copper, sodium, cadmium, lanthanides, gadolinium, technetium, calcium, potassium, chromium, magnesium, molybdenum and salts or complexes thereof), a toxin (e.g. a ligand targeted toxin, a protease activated toxin, melittin and a toxin-based suicide gene therapeutic) or a nanoparticle (e.g. a metal nanoparticle such as gold, iron, silver, cobalt cadmium selenide, titanium oxide) or a core-shell metal nanoparticle such as CdS/ZnS, CdSe/ZnS, CdSeICdS, and InAs/CdSe nanoparticle.

Preferably, wherein the number of TRAP rings in the TRAP-cage is between 6 and 60, preferably between 7 and 55, preferably between 8 and 50, preferably between 9 and 45, preferably between 10 and 40, preferably between 11 and 35, preferably between 12 and 34, preferably between 13 and 33, preferably between 14 and 32, preferably between 15 and 31, preferably between 16 and 30, preferably between 17 and 29, preferably between 18 and 28, preferably between 19 and 27, preferably between 20 and 26. Preferably the number of TRAP rings in the TRAP-cage is less than 40, preferably less than 35, preferably less than 30. Preferably the number of TRAP rings in the TRAP-cage is more than 6, preferably more than 10, preferably more than 15, preferably more than 20.

Preferably, the number of TRAP rings in the TRAP-cage is between 12 and 24.

Preferably, the number of TRAP rings in the TRAP-cage is about 24, preferably 24. Preferably, the number of TRAP rings in the TRAP-cage is about 12, preferably 12. Preferably, the number of TRAP rings in the TRAP-cage is about 20, preferably 20.

Preferably, opening of the cage is programmable. Preferably said specific conditions corresponds to the specific cleavage characteristic of the cross-linker.

Preferably, the programmable opening of the cage is dependent on selection of a molecular or atomic metallic cross-linkers which hold the TRAP-rings in place in the TRAP-cage.

Preferably, the specific cleavage characteristic of the molecular cross-linker is selected from the group comprising:
(i) a reduction resistant/insensitive molecular cross-linker, whereby the cage remains closed under reducing conditions;
(ii) a reduction responsive/sensitive molecular cross-linker, whereby the cage opens under reducing conditions; and
(iii) a photoactivatable molecular cross-linker whereby the cage opens upon exposure to light.

Preferably, the reduction resistant/insensitive molecular cross-linker can be selected from the group comprising: bismaleimideohexane (BMH) and bis-bromoxylenes. Preferably, the reduction responsive/sensitive molecular cross-linker can be selected from the group comprising: dithiobismaleimideoethane (DTME). Preferably, the photoactivatable molecular cross-linker can be selected from the group comprising: bis-halomethyl benzene and its derivatives including 1,2-bis-bromomethyl-3-nitrobenzene (o-BBN), 2,4-bis-bromomethyl-1-nitrobenzene (m-BBN) and 1,3-bis-bromomethyl-4,6-dinitro-benzene (BDNB).

Preferably, the molecular cross-linker is a homobisfunctional molecular moiety and its derivatives. Preferably, the homobisfunctional molecular cross-linker is bismaleimideohexane (BMH).

Preferably, the cage is resistant/insensitive to reducing conditions. Preferably the homobisfunctional molecular cross-linker is dithiobismaleimideoethane (DTME).

Preferably, the cage is responsive/sensitive to reducing conditions. Preferably, the molecular cross-linker is a bis-halomethyl benzene and its derivatives.

Preferably, the molecular cross-linker is selected from the group comprising, 1, 2-bis-bromomethyl-3-nitrobenzene (BBN), bis-bromoxylene and 1,3-bis-bromomethyl-4,6-dinitro-benzene (BDNB).

Preferably, the molecular cross-linker is photolabile by exposure to UV light.

Preferably, the cage according to the invention comprises a mixture of different programmable molecular cross-linkers.

Preferably, the interior surface of the TRAP-cage lumen is supercharged. Preferably the TRAP-cage with a supercharged lumen comprises a E48Q or a E48K mutation. Preferably the TRAP-cage with a supercharged lumen comprises a K35C/E48Q or a K35C/E48K mutation.

Preferably, the artificial TRAP-cage protein is modified to comprise any one or more of the following mutations selected from the group comprising K35C, E48Q, E48K R64S, K35C/E48Q, K35C/E48K, and K35C/R64S. Preferably the artificial TRAP-cage protein is modified to comprise a K35C mutation. Preferably the artificial TRAP-cage protein is modified to comprise a K35C mutation or a K35C/E48Q mutation or a K35C/E48K mutation.

Preferably, the artificial TRAP-cage protein is modified to comprise any one or more of the following mutations selected from the group comprising K35C, K35H, R64S, E48Q, E48K, K35C/R64S, K35H/R64S, S33C, S33H, S33C/R64S, S33H/R64S, S33C/K35H S33H/K35H, S33C/K35C, S33H/K35C, K35C/E48Q, K35C/E48K, K35H/E48Q, K35H/E48K, S33C/E48Q, S33C/E48K, S33C/E48Q, S33C/R64S/E48Q, K35H/S33C/R64S, S33C/R64S, S33H/K35H/R64S, S33C/K35C/R64S, K35C/R64S/E48Q, K35H/R64S/E48Q, K35H/R64S/E48K, S33C/R64S/E48Q, S33C/R64S/E48K, S33C/R64S/E48Q and S33C/E48K. Preferably the artificial TRAP-cage protein is modified to comprise a K35H/E48Q or a K35H/E48 mutation.

Preferably, the cage formation step of part (iii) for TRAP^{K35C E48Q} is performed in sodium bicarbonate buffer at pH 9-11.

Preferably, the cage formation step of part (iii) for TRAP^{K35C E48Q} is performed in sodium bicarbonate buffer at pH 10-10.5.

Preferably, the guest cargo can be loaded either pre or post assembly of the TRAP-cage.

Preferably, the genetic fusion of the guest cargo to an interior surface of the TRAP cage lumen of step (ii) is via N-terminus fusion of the guest cargo to an N-terminus of TRAP^{K35C} which faces into the interior surface of the lumen. Preferably the guest cargo is a genetically fused protein, preferably a fluorescent protein, preferably GFP, mCherry or mOrange. Preferably the genetically fused protein is interleukin-2 (IL-2) or Neoleukin-2/15 (NL-2).

Preferably, the SpyCatcher/SpyTag conjugation of the guest cargo to an interior surface of the TRAP-cage lumen of step (iii) wherein the SpyCatcher is introduced in a loop region of TRAP rings between residues 47 and 48, which faces to the interior when assembled into TRAP-cages and the guest cargo contains a SpyTag.

Preferably, enzymatic modification is via peptide ligase selected from the group comprising sortases, asparaginyl, endoproteases, trypsin related enzymes and subtilisin-derived variants and covalent chemical bond formation may include strain promoted alkyne-azide cycloaddition and pseudopeptide bonds.

If no cysteine is present in the biomolecule, or they are present but not available for the reaction, —SH group, preferably as a group of cysteine, may be introduced into the biomolecule.

Introduction of cysteine can be carried out by any method known in the art. For example, but not limited to, the introduction of the cysteine is performed by methods known in the art, such as commercial gene synthesis or PCR-based site-directed mutagenesis using modified DNA primers. Above-mentioned methods are known by the persons skilled in the art and ready-to use kits with protocols are available commercially.

—SH moiety may be introduced into the biomolecule also by modification of other amino acids in the biomolecule i.e. by site-directed mutagenesis or by solid phase peptide synthesis.

The subject matter of the invention is also a TRAP-cage produced by this method. These cages may have any of the features or properties as described in relation to the first aspect of the invention, above, or anything else described herein.

The subject matter of the invention is also use of the cage according to the invention, as defined above, in delivery of a cargo in a controlled period and to a desired location.

The subject matter of the invention is also use of any of the TRAP-cages described herein as a medicament.

The subject matter of the invention is also the use of any of the TRAP-cages described herein in treating a disease in a patient.

The subject matter of the invention is also a method of treating a patient, comprising administering the TRAP-cages described herein to said patient. The subject matter of the invention is also a method of treatment of an individual in need of therapy suffering from a condition selected from the group comprising cancer, vascular disease, cardiovascular disease, diabetes, infection, auto-immune condition, neurodegenerative and neurological disease, cellular senescence diseases, arthritis and respiratory disease, the method comprising administering a therapeutically effective amount of an artificial TRAP-cage bearing one or more internal guest cargoes selected from the group comprising a nucleic acid, an enzyme, a therapeutic agent, a small molecule, organic or inorganic nanoparticles, a peptide, a metal, an antigen, an antibody and toxin and fragments thereof of all the foregoing that are of therapeutic value.

The subject matter of the invention is also a method of vaccinating an individual in need of vaccination from a condition selected from the group comprising cancer, vascular disease, cardiovascular disease, diabetes, infection, auto-immune condition, neurodegenerative and neurological disease, cellular senescence disease, arthritis and respiratory disease, the method comprising administering a therapeutically effective amount of an artificial TRAP-cage bearing one or more internal guest cargo selected from the group comprising a nucleic acid, an enzyme, a therapeutic agent, a small molecule, organic or inorganic nanoparticles, a peptide, a metal, an antigen, an antibody and toxin and fragments thereof of all the foregoing that are of therapeutic value.

Preferably, the TRAP-cage therapeutic is administered via intranasal inhalation or injection.

### DETAILED DESCRIPTION OF THE INVENTION

Reference here to “TRAP protein” refers to Tryptophan RNA-binding attenuation protein, a bacterial protein. This protein can for example be isolated from wild type Geobacillus stearothermophilus, or other such bacteria. This protein can be isolated from various bacteria, but TRAP proteins which will work as described herein can be isolated from bacteria such as Alkalihalobacillus ligniniphilus, Anaerobacillus isosaccharinicus, Anoxybacillus caldiproteolyticus, Anoxybacillus calidus, Anoxybacillus pushchinoensis, Anoxybacillus tepidamans, Anoxybacillus tepidamans, Anoxybacillus vitaminiphilus, Bacillaceae bacterium, Bacillus alveayuensis,Bacillus alveayuensis, Bacillus sinesaloumensis, Bacillus sp. FJAT-14578, Bacillus sp. HD4P25, Bacillus sp. HMF5848, Bacillus sp. PS06, Bacillus sp. REN16, Bacillus sp. SA1-12, Bacillus sp. V3-13, Bacillus timonensis, Bacillus timonensis, Bacillus weihaiensis, Bacillus yapensis, Calidifontibacillus erzurumensis, Calidifontibacillus oryziterrae, Cytobacillus luteolus, Fredinandcohnia aciditolerans, Fredinandcohnia humi, Fredinandcohnia onubensis, Fredinandcohnia onubensis, Geobacillus genomosp. 3, Geobacillus sp. 46C-Ila, Geobacillus stearothermophilus, Geobacillus stearothermophilus, Geobacillus stearothermophilus, Geobacillus stearothermophilus, Geobacillus stearothermophilus, Geobacillus stearothermophilus, Geobacillus stearothermophilus, Geobacillus the rmodenitrificans NG80-2, Halobacillus dabanensis, Halobacillus halophilus, Halobacillus halophilus, Jeotgalibacillus proteolyticus, Litchfieldia alkalitelluris, Litchfieldia salsa, Mesobacillus harenae, Metabacillus, Metabacillus litoralis, Metabacillus sediminilitoris, Oceanobacillus limi, Oceanobacillus sp. Castelsardo, Omithinibacillus, Omithinibacillus bavariensis, Omithinibacillus contaminans, Omithinibacillus halophilus, Omithinibacillus scapharcae, Parageobacillus caldoxylosilyticus, Parageobacillus genomosp., Parageobacillus thermantarcticus, Parageobacillus thermantarcticus, Parageobacillus the rmoglucosidasius, Parageobacillus the rmoglucosidasius, Paucisalibacillus globulus, Paucisalibacillus sp. EB02, Priestia abyssalis, Priestia endophytica, Priestia filamentosa, Priestia koreensis, Priestia megaterium, Psychrobacillus glaciei, Salinibacillus xinjiangensis, Sutcliffiella cohnii, Thermolongibacillus altinsuensis.

Trp RNA-binding attenuation protein is a bacterial, ring-shaped homo 11-mer (see A. A. Antson, J. Otridge, A. M. Brzozowski, E. J. Dodson, G. G. Dodson, K. S. Wilson, T. Smith, M. Yang, T. Kurecki, P. Gollnick, which is hereby incorporated by reference), The structure of trp RNA-binding attenuation, protein can be seen in the literature (Nature 374,693-700 (1995), which is hereby incorporated by reference).

Suitably, the protein used herein is a modified version of wild-type TRAP isolated from Bacillus stearothermophilus. This is seen in Table 1:

TABLE 1

| Name | Protein sequence |
|---|---|
| Wild-type TRAP | MYTNSDFVVIKALEDGVNVIGLTRGADTRFHHSEKLDKGEVLIAQ |
| Bacillus | FTEHTSAIKVRGKAYIQTRHGVIESEGKK* |
| stearothermophilus | [SEQ ID NO: 1] |
| (PDB:1QAW) | |

The Wild-type TRAP Bacillus stearothermophilus gene sequence is seen in Table 2:

TABLE 2

| | | Gene ID |
|---|---|---|
| Name | Gene sequence | (from UniProt) |
| Wild-type TRAP | Atgtatacgaacagcgactttgttgtcattaaag | 58572467 |
| Bacillus | cgcttgaagacggagtgaacgtcattggattg | |
| stearothermophilus | acgcgcggggcggatacacggttccatcact | |
| | cggaaaagctcgataaaggcgaagtgttgat | |
| | cgcccagtttacagagcacacgtcggcgatta | |
| | aagtgagaggcaaggcgtatattcaaacgcg | |
| | ccatggcgtcattgagtcggaagggaaaaag | |
| | taa | |
| | [SEQ ID NO: 2] | |

Preferably, preparation of proteins is performed by biomolecule expression in a suitable expression system and purification of the expression product. Preferably with a modified version of the above Wild-type TRAP Bacillus stearothermophilus gene sequence.

TRAP proteins forms rings, herein “TRAP rings”, and rings are the natural state of TRAP proteins. Typically, as is the case for the Geobacillus Stearothermophilus proteins as demonstrated herein, TRAP monomer proteins spontaneously assemble into toroids or rings made from monomers.

Reference herein to a “TRAP-cage lumen” is the hollow interior of the TRAP-cage. It is separated from the external environment by TRAP rings which form the wall of the TRAP-cage where any holes in this wall are considered to separate the lumen form exterior environment by a flat plane between the edges of the TRAP-rings lining the hole.

TRAP-cages only form under particular conditions, for example as demonstrated herein with the presence of cysteines that can be crosslinked resulting in rings assembling into a cage. For example, as demonstrated herein, these will form with the presence of cysteine at position 35 (the result of a K35C mutation).

Reference herein to “TRAP ring” is synonymous with a TRAP building block, a subunit of the TRAP-cage complex or a TRAP monomer assembly. Reference herein to an “analog” of a particular protein or nucleotide sequence refers to a protein or nucleotide sequence having sufficient identity or homology to the protein or nucleotide sequence to be able to carry out the specified function, e.g. TRAP-cage formation under the conditions described herein, or encode a protein which is able to carry out the specified function, e.g. TRAP-cage formation under the conditions described herein.

To determine the percent identity/homology of two sequences, the sequence in question and a reference are aligned for optimal comparison purposes (e.g., gaps can be introduced in one or both of a first and a second amino acid or nucleic acid sequence for optimal alignment and non-homologous sequences can be disregarded for comparison purposes). A sequence may be determined an analog of a particular when it has preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the amino acids or nucleotides of the relevant lengths of the reference sequence. When the amino acid residues or nucleotides at corresponding amino acid positions or nucleotide positions are compared, when a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap.

Suitably, the TRAP protein comprises an amino acid sequence having at least 80%, preferably at least 85%, preferably at least 90%, preferably at least 95%, preferably at least 97% identity or homology to the amino acid sequence of SEQ ID NO: 1. Preferably, the TRAP protein comprises an amino acid sequence having at least at least 85% identity or homology to the amino acid sequence of SEQ ID NO: 1.

Reference herein to “TRAP-cage” refers to an assembled protein complex formed from multiple biomolecules, here multiple TRAP protein rings forming the complex. The TRAP protein rings can be linked together by crosslinkers, herein molecular cross-linkers. “Complex”, “assembly”, “aggregate”, are used alternatively in the description and means a superstructure constructed by the reaction between biomolecules. The amount of the units involved in the complex depends on the nature of the biomolecule. More specifically, it depends on the amount of the biomolecule and the amount of —SH groups present in the biomolecule.

TRAP protein is a suitable biomolecule model for the method of the invention. This is likely due to its high intrinsic stability, toroid shape, lack of native cysteine residues (for easier control of the conjugation process) and availability of a residue that can be changed to cysteines with the resulting cysteine being in a suitable chemical and spatial environment suitable for proper bond formation.

Reference herein to “programmable” is intended to convey that the TRAP-cages of the present invention have properties conferred on, or engineered into them that make them prone or susceptible or predisposed to behave in a particular and selected manner on exposure to specific environmental conditions or stimuli.

Reference herein to “open” is synonymous with the TRAP-cage, fracturing, leaking, fragmenting, breaking or generally allowing a cargo to escape from the interior of the cage.

Reference herein to “closed” is synonymous with the TRAP-cage remaining intact, unbreakable, impervious or generally remaining as a whole cage.

Reference herein to “bisfunctional” refers to a molecular crosslinker which has two functional groups, for example herein a molecule with two functional groups, where there is one functional group for each of the cysteine thiol groups to be crosslinked in order to connect TRAP rings in a TRAP-cage. Reference herein to “homobisfunctional” refers to a bisfunctional linker where the two groups are the same. Preferably, homobisfunctional linkers include bismaleimideohexane (BMH), dithiobismaleimideoethane (DTME), bis-halomethyl benzene and its derivatives, 2-bis-bromomethyl-3-nitrobenzene (BBN), bis-bromoxylene and 1,3-bis-bromomethyl-4,6-dinitro-benzene (BDNB).

“Molecular cross-linker” is a molecule that acts to connect units, subunits, molecules, biomolecules or monomers to other examples of the same via formation of one or more chemical bonds. Molecular crosslinkers are not single atoms linkers, which are distinct entities.

Reference herein in to “encapsulation” within the TRAP-cage is synonymous with enclosed, enveloped, contained or confined with the TRAP-cage.

Reference herein to a “guest cargo” refers to the biologic or whatever is encapsulated within the TRAP-cage.

The guest cargo could be a protein, preferably selected from the group comprising an enzyme (e.g. protease, a nuclease, hydrogenase, dehydrogenase, lipase, lyase, ligase, transferase, reductase, recombinase, nuclease acid modification enzyme. or other type of enzyme) an antigen, an antibody. Or the cargo is another type of protein biological macromolecule (e.g. a sterol, steroid or a fatty acid). Or the cargo is a lipid, a peptide (e.g. a peptide hormone, a cell membrane disrupting peptide, a T-cell-stimulating peptide or another type of peptides) a nucleic acid (e.g. DNA, designed DNA nanostructures including those designed using the DNA origami technique, DNAzymes, RNA, mRNA, miRNA, siRNA, tRNA single stranded RNA, double stranded RNA, RNAzymes), a small molecular cargo such as a drug, a peptide nucleic acids (PNA), a carbon-based structure (e.g. a fullerene or a buckminsterfullerene, a single walled carbon nanotube or a multi-walled carbon nanotube) a metal (e.g. iron, zinc, platinum, copper, sodium, cadmium, lanthanides, gadolinium, technetium, calcium, potassium, chromium, magnesium, molybdenum and salts or complexes thereof), a toxin (e.g. a ligand targeted toxin, a protease activated toxin, melittin and a toxin-based suicide gene therapeutic) or a nanoparticle (e.g. a metal nanoparticle such as gold, iron, silver, cobalt cadmium selenide, titanium oxide) or a core-shell metal nanoparticle such as CdS/ZnS, CdSe/ZnS, CdSeICdS, and InAs/CdSe nanoparticle.

The enzyme could be a protease is selected from the group comprising Bromelain, Botulinum toxin A, thrombin Factor VIIA, Protein C, TEV protease, serine proteases including the SB, SC, SE, SF, SH, SJ, SK, SO, SP, SR, SS, ST, PA, PB PC and PE superfamilies and the S48, S62, S68, S71, S72, S79, S81 families. Including, specifically Lon-A peptidase, Clp protease, lactoferin, nculeoporin 125, cysteine proteases including CA, CD, CE, CF, CL, CM, CN, CO, CP, PA, PB. PC, PD, and PE superfamililes and C7, C8, C21, C23, C27, C36, C42, C53 and C75 families including specifically papain, cathepsin K, calpain, separase, adenain, sortase A and Hedhehog protein, aspartic proteases including AA, AC, AD, AE and AF superfamilies including specific examples as follows, BACE1, BACE2, Cathespin D, CathespinE Chymosin, Napsin-Ad, Nepenthesin, Pepsin, Presenilin, plasmepsins, threonine proteases including PB and PE superfamilies including specifically orhithine acyltransferase, glutamic proteases including G1 and G2 superfamilies, metalloproteinases including metalloexpeptidases and metalloendopeptidases.

The enzyme could be nuclease is selected from the group comprising endonucleases e.g. deoxcyribonuclease I; human endonuclease V, CRISPR associated proteins (including Cas9, Cas12, Cas13) with or without associated nucleic acids including guide RNA; AP endonuclease; flap endonuclease

The protein could be another type of enzyme, for example SUMO Activating Enzyme E1, a DNA repair enzymes e.g. DNA ligase, a DNA methyltransferases e.g. the m6A, m4C and m5C classes, a ten-eleven translocation methylcytosine dioxygenase, early growth response protein 1 (EGR1), Oxoguanine glycosylase, a Caspase e.g. E3 ubiquitin ligases including including pVHL,CRBN, Mdm2, beta-TrCP1, DCAF15, DCAF16, RNF114, c-IAP1, or an E1 ligase, an E2 ligase, DNA glycosylase, ora toxin e.g. ricin toxin A chain, Diptheria toxin and fragemnts thereof, a pore-forming toxins e.g. exotoxin A, α-hemolysin, Gyr-I, Myeloid cell leukemia 1 (Mcl-1), a DNA polymerase including DNA polymerase β, polymerase δ and polymerase ϵ or an Enzyme replacement therapy enzyme e.g, Agalsidase beta, Agalsidase alfa, Imiglucerase, Taliglucerase alfa, Velaglucerase alfa, Alglucerase, Sebelipase alpha, Laronidase, Idursulfase, Elosulfase alpha, Galsulfase, Alglucosidase alpha.

The cargo could be something that could act recognised as an antigen, e.g. SARS-CoV-2 spike protein full length, SARS-CoV-2 spike protein, receptor binding domain, SARS-CoV-2 spike protein, peptides thereof, SARS-CoV-2 spike protein full length, SARS-CoV-2 spike protein, receptor binding domain, SARS-CoV-2 spike protein, peptides thereof, AARS-CoV-2 non-spike structural proteins, SARS-CoV-2 non-spike structural proteins, peptides thereof, SARS-Cov-2 genome encoded proteins or parts thereof, Respiratory Syncytial Virus spike protein full length, Respiratory Syncytial Virus spike protein, receptor binding domain, Respiratory Syncytial Virus spike protein, peptides thereof, Respiratory Syncytial Virus spike protein full length, Respiratory Syncytial Virus spike protein, receptor binding domain, Respiratory Syncytial Virus spike protein, peptides thereof, Respiratory Syncytial Virus non-spike structural proteins, Respiratory Syncytial Virus non-spike structural proteins, peptides thereof, Respiratory Syncytial Virus genome encoded proteins or parts thereof, Lassa virus spike protein full length, Lassa virus spike protein, receptor binding domain, Lassa virus spike protein, peptides thereof, Lassa virus spike protein full length, Lassa virus spike protein, receptor binding domain, Lassa virus spike protein, peptides thereof, Lassa virus non-spike structural proteins, Lassa virus non-spike structural proteins, peptides thereof, Lassa virus genome encoded proteins or parts thereof, Epstien-Barr virus spike protein full length, Epstien-Barr virus spike protein, receptor binding domain, Epstien-Barr virus spike protein, peptides thereof, Epstien-Barr virus spike protein full length, Epstien-Barr virus spike protein, receptor binding domain, Epstien-Barr virus spike protein, peptides thereof, Epstien-Barr virus non-spike structural proteins, Epstien-Barr virus non-spike structural proteins, peptides thereof, Epstien-Barr virus genome encoded proteins or parts thereof, Dengue Fever virus structural proteins N, M or E, Dengue Fever virus structural proteins N, M or E, peptides thereof, Dengue Fever virus structural proteins N, M or E, portions thereof, cytomegalovirus proteins, portions therof and derived peptides including capsid proteins, tegument proteins, polymerases and other proteins encoded by the viral genome, Influenza Virus HA protein full length, Influenza Virus HA protein, receptor binding domain, Influenza Virus HA protein, peptides thereof, Influenza Virus non-HA structural proteins, Influenza Virus non-HA structural proteins, peptides thereof, Influenza Virus genome encoded proteins or parts thereof.

The cargo could be an antibody e.g. Anti-p53 antibody, an anti-mutant p53 antibody, an Anti-JAK mAb e.g. Tofacitinib and baricitinib, an Interleukin inhibitor e.g. tocilizumab, secukinumab and ustekinumab, an Anti-CD20 mAbs e.g. Rituximab, ofatumumab and ocrelizumab, an Anti-TNF mAb e.g. Infliximab, adalimumab and golimumab, an Anti-IgE mAb e.g. Omalizumab, Haemopoietic growth factors such epoetin, Anti-PD1 and PDL-1 mAb such Keytruda, Anti-CTLA4 mAb e.g. ipilimumab, Anti-1L2 antibodies, Anti-1112 antibodies, Anti-I115 antibodies, Anti-TGFBeta antibodies, Anti-angiogenesis mAb e.g. Avastin, Antagonist mAb of the A2A and A2B receptors, Anti-Her2 mAb e.g. Trastuzumab, Antibody dependent conjugates, Anti-EGFR mAb, Anti-VEGFR mAb, Anti-CD52 mAb e.g. Alemtuzumab, anti-BAFF mAb e.g. Belimumab, Anti-CD19 mAs e.g. Blinatumomab, Anti-CD30 mAb e.g. Brentuximab vedotin Anti-CD38 mAb e.g. Daratumumab, Anti-VEGFR2 mAb e.g. Ramucirumab or an Anti-IL6 mAb e.g. Siltuximab.

The protein could be another type of protein, for example Target-of-Rapamycin (TOR), GATA transcrition factor Gaf1 (Gaf one), A TALE (Transcription activator-like effectors) protein, a Zinc finger protein, a Tumor suppressor proteins including those involved in control of gene expression e.g. p16, signal transducers e.g. (TGF)-I3; checkpoint control protein e.g. BRCA1, proteins involved in cell adhesion e.g. CADM1, DNA repair proteins e.g. p53, a transcription factor e.g. Yamanaka factors (Oct3/4, Sox2, Klf4, c-Myc), cytochrome c, BCL proteins including Bcl-2 (B-cell lymphoma 2), transcriptional control proteins e.g. NF-KB, a Cytokine including chemokines, interferons, interleukins Including interleukin-2 and artificial versions thereof), lymphokines, and tumour necrosis factors, a Heat shock protein including heat shock beta-one protein, a Growth factor e.g. GDF11, ubiquitin, a DNA double-strand break repair protein e.g. DNA ligase IIla, a PCSK9 inhibitor e.g. evolocumab and alirocumab, a Brain-derived neurotrophic factor (BDNF) or Inhibitors of IL-5 e.g. mepolizumab and reslizumab.

The cargo could be another type of biological macromolecule (e.g. a sterol, steroid or a fatty acid. The sterol may be cholesterol. The steroid may be progesterone. The fatty acid may be a saturated fatty acid eg. Caprylic acid, capric acid, lauric acid, myristic acid, palmitic acid, stearic acid, arachidic acid, behenic acid, lignoceric acid, cerotic acid or an unsaturated fatty acid e.g. Myristoleic acid, Palmitoleic acid, Sapienic acid, Oleic acid, Elaidic acid, Vaccenic acid, Linoleic acid, Linoelaidic acid, α-Linolenic acid, Arachidonic acid, Eicosapentaenoic acid, Erucic acid, Docosahexaenoic acid

The cargo could be a lipid, such as phospholipids e.g. phsophotdiylcholine, Phosphatidic acid (phosphatidate) (PA), Phosphatidylethanolamine (cephalin) (PE), Phosphatidylserine (PS), Phosphatidylinositol (PIO, Phosphatidylinositol phosphate (PIP), Phosphatidylinositol bisphosphate (PIP2) and Phosphatidylinositol trisphosphate (PIPS), (Sphingomyelin) (SPH)Ceramide phosphorylethanolamine (Sphingomyelin) (Cer-PE).

The cargo could be a peptide, such as a peptide hormone, a cell membrane disrupting peptide, a T-cell-stimulating peptide, or another type of peptide. The peptide hormone may be adrenocorticotropic hormone (ACTH), amylin, angiotensin, atrial natriuretic peptide (ANP), calcitonin, cholecystokinin (CCK), gastrin, ghrelin, glucagon, growth hormone, follicle-stimulating hormone (FSH), insulin, leptin, luteinizing hormone (LH), melanocyte-stimulating hormone (MSH), oxytocin, parathyroid hormone (PTH), prolactin, renin, somatostatin, thyroid-stimulating hormone (TSH), thyrotropin-releasing hormone (TRH), vasopressin, also called arginine vasopressin (AVP) or anti-diuretic hormone (ADH) or vasoactive intestinal peptide (VIP). The cell membrane disrupting peptide may be melittin. The T-cell-stimulating peptide may be an antigen such as the portions of antigen proteins described above. Another type of peptide may be Microcin B-17 and derivatives, Albicidin and derivatives, Peptide inhibitors of Myeloid cell leukemia 1 (mcl-1), pepstatin and derivatives thereof.

The cargo could be a small molecular cargo, such as antibiotic molecules e.g. a macrolide antibiotic, nicotinamide adenine dinucleotide (NAD+), nicotinamide mononucleotide, a chloresterol absorption inhibitor e.g. ezetimibe, a Fibrate e.g. gemfibrozil, bezafibrate and cipofibrate, HMG-CoA Reductase Inhibitor, Ranolazine, Ivabradine, a Nitrate such as glyceryl trinitrate, an Endothelin antagonist such as Bosentan, Hydralazine, Minoxidil, a Calcium channel blocker e.g. amlodipine, nifedipine, verapamil, diltiazem, an Angiotensin antagonist e.g. losartan, valsartan, candesartan and irbesartan, an ACE inhibitor e.g. captopril, enalapril, lisinopril, Digoxin, an Adenosine receptor agonist, a class IV Antidysrhythmic e.g. Verapamil Class III Antidysrhythmic e.g. Amiodarone, Class II Antidysrhythmic e.g. bisoprolol, esmolol and propranolol, Class I Antidysrhythmic e.g. Flecainide and Disopyramide, Anti-histamine e.g. Promethazine, cyclizine and Cetirizine, Glucocorticoid e.g. Prednisolone, dexamethasone and hydrocortisone, an Antiproliferative Immunosuppressant, an Calcineurin Inhibitor e.g. ciclosporin, an Uricosuric Agent e.g. Allopurinol and flebuxostat, a DMARD, a COX-2 Inhibitor e.g. Celecoxib, etoricoxib and parecoxib, a NSAID, a DOPA Decarboxylase Inhibitor e.g. Carbidopa or benserazide, a Selective B3-Adrenoceptor agonist, an a1-receptor agonist, a B1 receptor agonist e.g. Dobutamine, an al receptor antagonist e.g. prazosin, doxazosin and tamsulosin, a B2 receptor agonist e.g. salbutamol and terbutaline, a Nicotinic Partial Agonist e.g. Varenicline, a Peripheral Anticholinesterases e.g. Neostigmine, a Neuromuscular blocker e.g. panucuronium, vecuronium and rocuronium, a Bladder control drug e.g. oxybutynin and tolterodine, an Anti-metabolite e.g. folate antagonists, pyrimidine analogues and purine analogues, an Alkylating agent, an anti-fungal drug e.g. Grisofluvin, caspofungin and terbinafine, an anti-fungal antibiotic e.g. Amphotericin and nystatin, an Artemisinin Derivative e.g. artesunate and artemisinin, a Folate inhibitor e.g. proguanil, Primaquine, a Blood schizonticide e.g. chloquine and quinine, a Neuraminidase inhibitor e.g. Oseltamivir and zanamivir, a DNA Polymerase Inhibitor e.g. Aciclovir and glanciclovir, a Protease inhibitor e.g. Darunavir and ritanovir, a Reverse transcriptase inhibitor e.g. nevirapine and efavirenz, an Antiepileptic drug e.g. Carbamezepine, gabapentin, and pregabalin, a Tricyclic antidepressant e.g. amitriptyline nortriptyline and desipramine, an Opioid, a AMPA receptor Blocker e.g. Topiramate, a Barbiturate, a Benzodiazepin e.g. Lorazepam, midazolam and diazepam, a sodium channel inhibitors e.g. Carbamezepine, oxcarbazepine and phenytoin, a drug for bipolar disease e.g. lithium, a dopamine reuptake inhibitor e.g. Bupropion, a Monoamine oxidase inhibitor e.g. phenelzine, isocarboxcazid and moclobemide, a Noradrenaline reuptake inhibitor e.g. reboxetine and maprotiline, a SNRI e.g. venlafaxine, duloxetidne and desvenlafaxine, a SSRI e.g. fluoxetine, paroxetine, citalopram, escitalopram and sertraline, a Tricyclic e.g. imipramine and clomipramine, an Anti-pysychotic e.g. amisulpride and supiride, a Partial serotonin agonist, a NMDA receptor antagonist e.g. memantine, a Cholinesterase inhibitor e.g. donepezil, rivastigmine and galantamine, a Monoxidase inhibitor e.g. selegiline and rasagiline, a COMT inhibitors such as entacapone and tolcapone, a Dopamine agonists e.g. pramipexole and rotigotine, a Phosphodiesterase Type V inhibitor e.g. sildenafil and tadalafil, a Uterine stimulant e.g. misoprostal, ergometrine and oxytocin, a GnRH analogue and inhibitors, an Alpha-glucosidase inhibitor, a SGLT-2 inhibitor e.g. canagliflozin and empagliflozin, a Dipeptidyl Petidase Inhibitor e.g. sitagliptin, saxagliptin and linagliptin, a Proton pump inhibitor e.g. Omeprazole, lansoprazole and pantoprazole, an Inhaled glucocorticoid e.g. neclometasone and budesonide, a Inhaled muscarinic antagonist e.g. tiotropium and glycopyrronium, a Leukotriene antagonist e.g. montelukast, a Beta2-receptor agonist e.g. almetrol and formoterol, an Anticoagulant e.g. dabigratran, heparin and apixaban, a STING antagonist, an Inflamasome inhibitor, a Targeted oncology drug, a Protein kinase inhibitor, a Cell cycle inhibitor, a PROTAC and other promoter of protein degradation, PARP inhibitor e.g. Niraparib, a ALK inhibitor e.g. Alectinib, a HDAC inhibitor e.g. Belinostat, a MEK inhibitor e.g. Cobimetinib, a BRAF inhibitor e.g. Dabrafenib, EGFR inhibitor e.g. Erlotinib, a mTOR inhibitor e.g. Everolimus, a HER2 inhibitor e.g. Lapatinib, a FLT3 kinase inhibitor e.g. Midostaurin, a JAK inhibitor e.g. Tofacitinib or a BCL2 inhibitor e.g. Venetoclax.

“Unit”, “subunit”, “molecule”, “biomolecule”, “monomer” are used alternatively in the description and means one molecule which connects to another molecule for the complex formation.

Reference herein to a “Reduction resistant/insensitive molecular cross-linker” is reference to a cross-linker which is not cleaved by reduction reaction such as that typically seen when a disulphide bond is cleaved by a reducing agent. These cross-linkers are stable under conditions that would result in breaking of reduction sensitive bonds. These bismaleimideohexane (BMH) and bis-bromoxylenes.

Reference herein to a “Reduction responsive/sensitive molecular cross-linker” is reference to a cross-linker which is cleaved by reduction reaction such as that typically seen when a disulphide bond is cleaved by a reducing agent. These cross-linkers are not stable under conditions that would result in breaking of reduction sensitive bonds. These include dithiobismaleimideoethane (DTME).

Reference herein to a “Photoactivatable molecular cross-linker” is reference to a cross-linker that is photoreactive or sensitive to light, i.e. one that will be cleaved when exposed to light. This light can be UV or other such light of a specific range of wavelengths. These include ,2-bis-bromomethyl-3-nitrobenzene (o-BBN), 2,4-bis-bromomethyl-1-nitrobenzene (m-BBN) and 1,3-bis-bromomethyl-4,6-dinitro-benzene (BDNB).

Reference herein to the lumen of a TRAP cage being “supercharged” means that the lumen-facing surface of the cage undergoes a net change in charge equivalent to at least +1 or −1 per TRAP-ring compared to the unmutated (wild type) ring. Thus, a 24-ring TRAP-cage would, when supercharged, carry a minimum change in charge of −24 or +24 compared to the non-supercharged variant

Moreover, following abbreviations have been used: TRAP (trp RNA-binding attenuation protein), GFP (green fluorescence protein), PTD4 (protein transduction domain), CPP (cell penetrating peptide), SDS-PAGE (sodium dodecyl sulfate-polyacrylamide gel electrophoresis), TEM (transmission electron microscopy), DMEM (Dulbecco's Modified Eagle Medium), FBS (foetal bovine serum).

Transport of molecular cargoes to cells is desirable for a range of applications including delivery of drugs, genetic material or enzymes. A number of nanoparticles have been employed to achieve this including liposomes, virus-like particles, non-viral protein cages, DNA origami cages and inorganic nanoparticles, each with their own advantages and disadvantages. Protein cages are a promising approach as demonstrated by viruses in nature which are able to deliver genetic material to cells, often with high efficiency and specificity.

Artificial cages are constructed by proteins which do not naturally form cage structures and in which interactions between constituent proteins may be modified to promote their assembly. The advantage of using such an approach is that the resulting cages can be given properties and capabilities that may not be available or feasible in naturally occurring forms. To date a number of artificial protein cages have been produced including tandem fusions of proteins with 2- and 3-fold rotational symmetries able to form a 12-subunit tetrahedral cage, a nanocube structure of 24 subunits with octahedral symmetry, a 60-subunit icosahedral cage structure that self-assembles from trimeric protein building blocks, and co-assembling two-component 120-subunit icosahedral protein complexes comparable to those of small viral capsids as well as designed peptides able to form networks that close to form cages. Several examples exist where artificial protein cages have been filled with various cargoes including siRNA, mRNA² and fluorescent dyes. However, only a handful of cases have demonstrated delivery of cargo to cells by artificial cages. To the best of our knowledge, delivery of protein/therapeutic cargoes to cells mediated by artificial protein cages (as opposed to natural cages) has not previously been demonstrated.

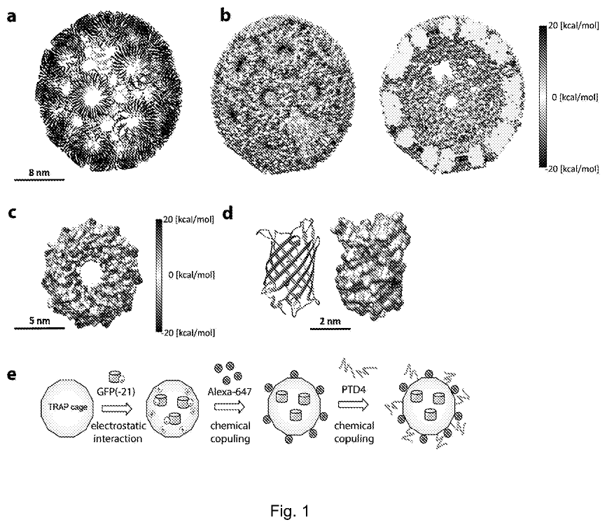

We previously produced an artificial protein cage using a building block consisting of the naturally occurring ring-shaped protein, TRAP (trp RNA-binding attenuation protein) referred to as TRAP-cage (FIG. 1a)¹. In nature, TRAP is involved in control of tryptophan synthesis and has been well characterised structurally and biochemically. It has also been used as a versatile building block in bionanoscience. TRAP-cage consists of 24 TRAP rings forming an approximately 22 nm diameter, 2.2 MDa hollow sphere with a lumen roughly 16 nm in diameter. Each TRAP ring in the cage is bound to 5 TRAP ring neighbours and the structure contains 6 square holes approximately 4 nm in diameter. Unusually, compared to other natural and most artificial cages, the ring subunits in the cage are held together not by a network of protein-protein interactions. Instead, single gold(I) ions bridge opposing sulphurs of the cysteine residues between rings in proteins where naturally occurring lysine at position 35 is replaced with cysteine. The cysteines of ten of the 11 monomers of each ring in the cage are bridged in this way with an eleventh remaining unbridged and available to react, e.g. with maleimide-labelled dyes.

TRAP-cage is extremely stable, able to survive temperatures of 95° C. for at least 3 hours, and high levels of denaturing agents such as urea. Despite this high stability TRAP-cage breaks apart readily in the presence of low concentrations of reducing agents including the cellular reducing agent glutathione. This feature raises the prospect that the TRAP-cage may have utility as a system for delivering cargo to cells, as it can be expected to retain its structure, protecting cargo until entering cells where intracellular reducing agents will result in disassembly and subsequent cargo release.

We have shown that TRAP-cage can be deliberately filled with protein cargo, and we use a negatively supercharged variant of green fluorescent protein, GFP(−21), as an exemplar molecule. We also show that TRAP-cage can be used to deliver such cargoes to the interiors of human cells. This cell-penetration is itself controllable as it only occurs if the surface of TRAP-cage is modified, e.g. by cell-penetrating peptide. The results are a first step towards development of TRAP-cage as a potentially useful tool for delivering medically relevant cargoes to cells and more generally demonstrates the potential for artificial protein-cage systems as therapeutic agents.

Here we show that TRAP-cage can be used to deliberately encapsulate a protein cargo and deliver it to cell interiors. TRAP-cages employed either in unmodified form or externally decorated, showed no significant effects on cell viability.

In the first case, filling with cargo was achieved using our previously developed TRAPcage¹ having positively charged patches on its interior, to capture negatively supercharged GFP electrostatically through diffusion into the cage. Attempts to deliver filled cages to cells showed no evidence of penetration of TRAP-cages into cells if they were undecorated. In contrast, attachment of cell penetrating peptide (CPP) PTD4 to the exterior of TRAP-cages resulted in significant penetration into cell interiors.

A small number of previous works on artificial protein cage-mediated delivery of cargo to cells have demonstrated success for non-protein cargoes. Notably, it has been shown that an artificial protein cage loaded with siRNA can be taken up by different mammalian cells and release its cargo to induce RNAi and knockdown of target gene expression³. In this case, the high gene silencing efficiency together with low toxic effects indicated that a protein cage carrier has potential as a therapeutic delivery system. Encapsulation of protein cargoes within artificial protein cages has previously been demonstrated. However, these cages were not shown to be able to directly deliver their cargo to cells, instead multiple copies of the cages were themselves used as cargoes within lipid envelopes made in cells and purified as enveloped protein nanocages' (EPNs) where the lipid envelope was derived from the host cell membrane. The EPNs were able to deliver the cages meaning that entry to cells was achieved by the enveloping, host-derived membrane, not the protein cage.

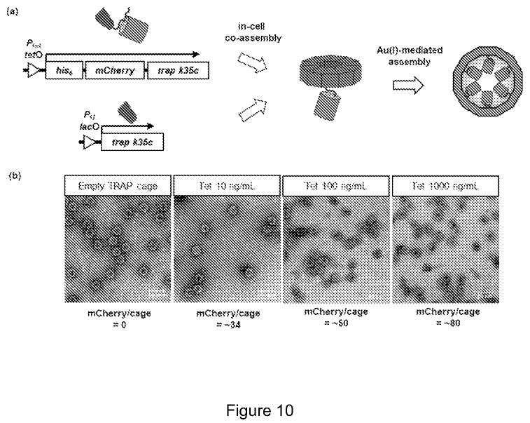

Given the overall high stability of TRAP-cage but its proven ability to disassemble in the presence of cellular reducing agents¹ it would be interesting to know if cages readily break apart once inside cells. The change in relative signal strengths of TRAP-cage associated Alexa-647 versus GFP once in the cell is suggestive of intracellular breakup of the cage and release of the cargo. A possible explanation is that when Alexa647 and GFP are in close proximity to each other due to association with TRAP-cage, the GFP fluorescence may be decreased due to a quenching effect from the dye. Once GFP is released by TRAP-cage disassembly, average GFP to Alexa-647 distances become larger, resulting in an increase in detected GFP fluorescence. This possibility is supported by the observation that the signal from intracellular GFP is visibly brighter when it is delivered using TRAP-cage lacking Alexa-647 (FIG. 10).

Overall, the work presented herein offer a first demonstration of protein delivery to cells mediated by artificial protein cages. The cargo-filling efficiency demonstrated was quite low and this could be addressed by modifying TRAP-cage further such that it carries a higher density of positive charge within the cage interior.

Further, demonstrated is loading of functional protein cargoes into TRAP-cage using genetic fusion of the cargo to the lumen-facing N-terminus of TRAP monomers. A patchwork expression approach allows mixed expression of TRAP monomers with the fusion or without allowing control of the amount of cargo such that it does not exceed the capacity of the cage. In this way TRAP-cage bearing mCherry cargo and bearing a mix of mCherry and mOrange cargo were demonstrated and the presence of cargoes confirmed.

Further, the genetic fusion approach was adapted to provide multiple copies of a lumen-facing SpyCatcher protein and it was demonstrated that this was capable of capturing functional proteins, a green fluorescent protein (GFP) and a computationally designed interleukin-2 mimicry (Neoleukin-2/15, NL-2) (Silva D A, et al. Nature, 2019, 565, 186-191), bearing a SpyTag. We further showed that interaction of NL-2 with its target cellular receptor was significantly inhibited by encapsulation in the TRAP cage, the cargo became functional in a similar level to free NL-2 when the TRAP-cage was opened by an externally applied trigger.

Alternatively, different methods of cargo capture (such as covalent attachment) could be explored, as described for other protein cages.^{4,5} Additionally we anticipate further modification of TRAP-cage both to increase targeting specificity and to extend the range and usefulness of encapsulated cargo. Finally, future studies will be required to pinpoint and track both the precise intracellular location of TRAP-cages and their quaternary state. According to an aspect, the cages as described herein may be used as medicaments. This could be in a of treating a patient, such as comprising administering a cage as described herein to a patient, or the cages as described herein for use in treating a disease in a patient. This particularly may be a cage designed to carry cargo and disassemble in presence of reducing agents for intracellular delivery. These cages may be administered along with or in the presence of a pharmaceutically acceptable carrier, adjuvant or excipient. The cargo that the cages for use as a medicament or for treating patients will be of benefit to said patient. For example, as drug delivery systems (DDS)—for active molecules (especially biological macromolecules such as RNA, DNA, peptides and proteins). They provide advantages ast as biological macromolecules are often easily disrupted or digested by conditions such as those found in vivo. Biological macromolecules are too big to diffuse out of the holes in TRAP, being a large protein, TRAP-cage can sustain significant changes without disrupting overall structure. This means that it can be modified to capture therapeutic cargoes and simultaneously be modified, on the exterior to target therapeutic targets. Programmable linkers can be used which cleave in a desired situation that correlates with arrival at site of action. For example, light could be shone on the target site to cleave open photocleavable TRAP-cages. If TRAP-cages penetrate cells, those held together by reducible linkers will spontaneously open up and release cargo as the cytoplasm of the cell is highly reducing. Cages could also be used in conjunction with vaccines or acting as vaccines, where antigens (i.e. peptides) which are expected to stimulate a T-cell response are captured inside the TRAP-cage and then targeted at to T-cells, followed by triggered opening.

### BRIEF DESCRIPTION OF THE FIGS.

FIG. 1. TRAP-cage protein. (a) Structure of TRAP-cage (PDB:6RVV) with each TRAP ring shown a different colour. Gold atoms are shown as yellow spheres. (b) Surface representation of TRAP-cage exterior (left) and interior (right) coloured by charge distribution. (c) Surface view of a single TRAP-ring with the face that points into the interior cavity shown, coloured according to charge. (d) Negatively supercharged GFP(−21) shown in cartoon representation (left) and surface view coloured according to charge (right). (e) Scheme of TRAP-cage encapsulation with GFP(−21) and external modifications with Alexa-647 dye and PTD4 peptide.

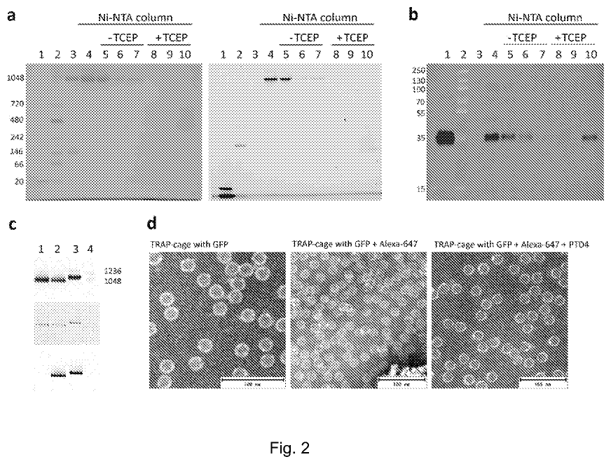

FIG. 2. Filling and deco ration of TRAP-cage. (a) Native PAGE gels showing purified TRAP-cage incubated with His-tagged GFP(−21) after passing through a Ni-NTA column in the absence (−TCEP) or presence (+TCEP) of TCEP. Lane 1: GFP(−21) positive control; 2: molecular weight marked for native PAGE; 3: empty TRAP-cage; 4: input (TRAP-cage with GFP(−21)); 5 and 8: flow-through; 6 and 9: wash; 7 and 10: elution. Collected fractions were stained for protein (left) or analysed by fluorescence detection (right, exct. 488 nm). (b) Collected fractions were subjected to SDS-PAGE followed by Western blot with anti-GFP detection. Lane 1: GFP(−21) positive control; 2: molecular weight marker for SDS-PAGE; 3: empty TRAP-cage; 4: input (TRAP-cage with GFP); 5 and 8: flow-through; 6 and 9: wash; 7 and 10: elution. (c) Native PAGE gels showing encapsulation of GFP(−21) by unmodified TRAP-cage or TRAP-cage externally modified by Alexa-647 and PTD4. Lane 1: TRAP-cage with GFP(−21); 2: TRAP-cage with GFP(−21) decorated with Alexa-647; 3: TRAP-cage with GFP(−21) decorated with Alexa-647 and PTD4; 4: molecular weight marker for native PAGE. Gels were stained for protein (upper panel) and analysed by fluorescence detection of

GFP (middle panel, exct. 488 nm) and Alexa-647 (bottom panel, exct. 647). (d) Negative stain transmission electron microscopy of TRAP-cage with GFP(−21) (left panel); TRAP-cage with GFP(−21) decorated with Alexa-647 (middle panel); TRAPcage with GFP(−21) decorated with Alexa-647 and PTD4 (right panel).

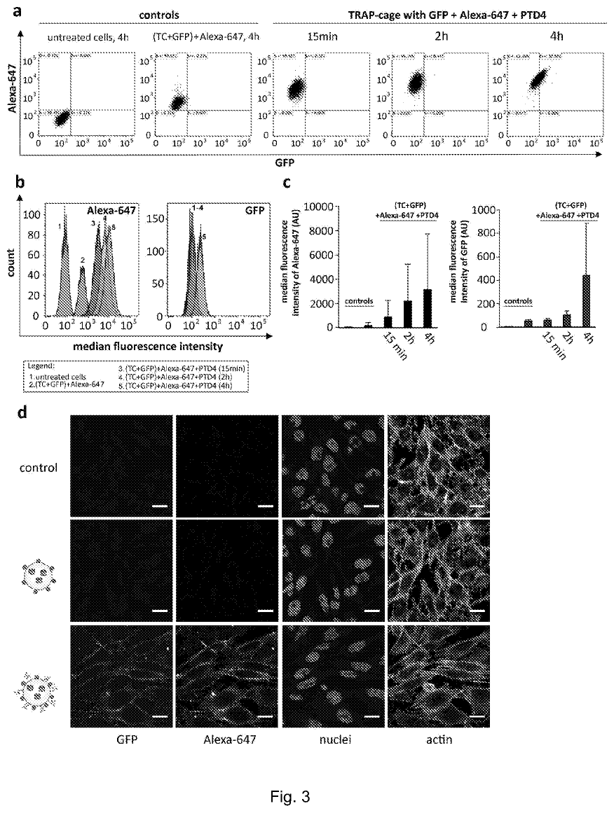

FIG. 3. Delivery of TRAP-cage carrying GFP(−21) to MCF-7 cells. (a) Representative flow cytometry dot plots of MCF-7 cells after 4 h treatment with Alexa-647 labelled TRAP-cage carrying GFP(−21) (denoted as (TC+GFP) +Alexa-647) and TRAP-cage with GFP(−21) labeled with Alexa-647 and PTD4 peptide (denoted as (TC+GFP) +Alexa-647+PTD4) for 15 min, 2 h and 4 h. The x-axis and the y-axis show the fluorescent intensities of GFP and Alexa-647, respectively. Untreated cells were used as the negative control. (b) Representative red and green fluorescence overlay histogram plot of MCF-7 cells from the same experiment. (c) Median fluorescence intensity of Alexa-647 and GFP positive cells treated with TRAP-cage carrying GFP and decorated with Alexa-647 or decorated with both Alexa-647 and PTD4 after 15 min, 2 h and 4 h incubations. Data are normalized to untreated cells and based on three independent experiments. Controls: 1: untreated cells; 2: cells incubated with (TC+GFP)+Alexa-647. (d) Confocal microscopy images of untreated cells (control cells, upper row), cells incubated with TRAP-cage filled with GFP(−21) and labeled with Alexa-647 only (middle row): cells incubated with TRAP-cage filled with GFP(−21) and labeled with Alexa-647 and PTD4 (bottom row). Actin filaments were stained with phalloidin conjugated to Alexa-568 and nuclei were stained with DAPI. Green channel—GFP; red channel—Alexa-647; blue channel—DAPI; grey channel—Alexa-568; (scale bar: 10 μM).

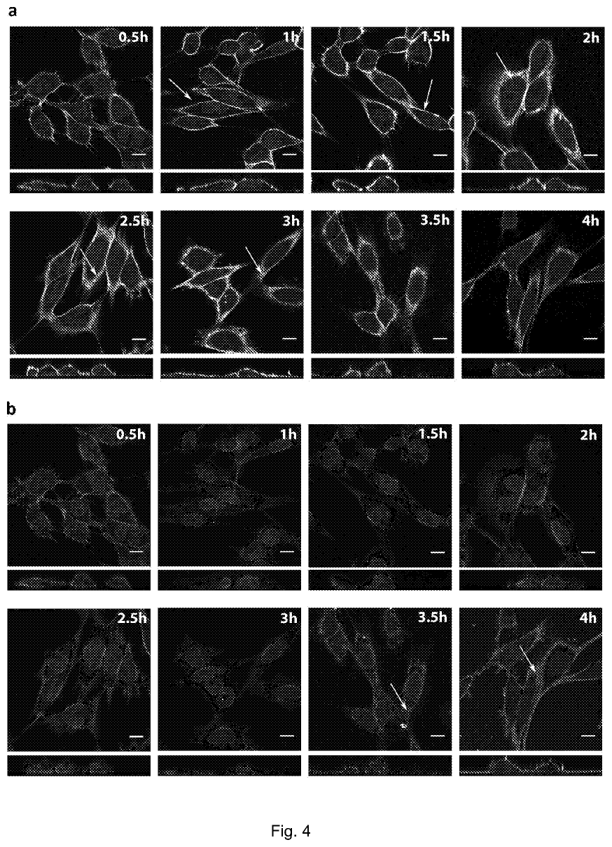

FIG. 4. Tracking TRAP-cage and GFP(−21) in MCF-7 cells. Confocal microscopy merged images of cells incubated with TRAP-cage carrying GFP(−21) decorated with Alexa-647 and PTD4 and fixed at different time points. Actin was stained with phalloidin conjugated to Alexa-568 whereas DAPI was used for nuclear staining; (scale bar: 10 μM). Rectangular images beneath each main image are representative orthogonal views in the yz axis. (a)—images with red channel maximal projection; (b)—images with green channel maximal projection.

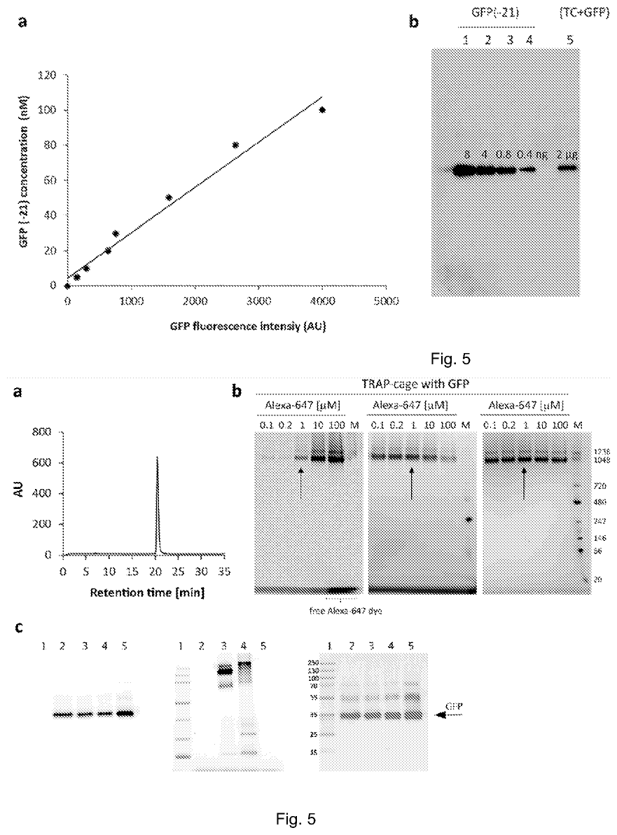

FIG. 5. Estimating the number of His-tagged GFP(−21) molecules in the TRAP-cage. (a) Standard curve obtained from fluorescence measurements of GFP(−21) protein, with the concentration range from 0-100 nM. Fitted with equation: y=0.0258x+4.4; R²=0.9786. (b) Western blot used for band densitometry analysis. Lanes 1-4: GFP(21); lane 5: TRAP-cage loaded with GFP(−21) (denoted as (TC+GFP)).

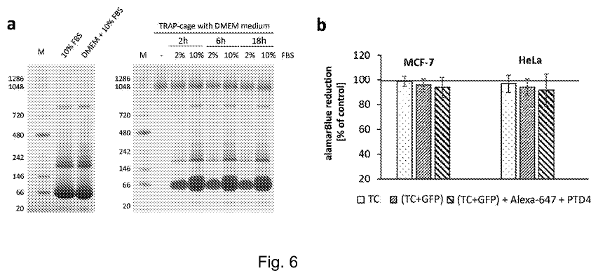

FIG. 6. External decoration of TRAP-cage with GFP(−21) (a) RP-HPLC chromatogram showing purified PTD4 peptide used to decorate TRAP-cage filled with GFP(−21). (b) Native PAGE gels showing TRAP-cage carrying GFP(−21) after titration of Alexa-647 in the conjugation reaction. Gels were analysed by fluorescence detection of Alexa647 (left panel, exct. 647) and GFP (middle panel, exct. 488 nm) and stained for proteins (right panel). Arrows show optimal decoration conditions used in further experiments. (c) SDS-PAGE gel comparing TRAP-cages carrying GFP(−21) either with no decoration, decorated with Alexa-647 or decorated with both Alexa-647 and PTD4. Left: detection at 488 nm; middle: detection at 647 nm; right: Western blot of the same samples detected with anti-GFP antibody. Lanes: 1: molecular weight marker for SDSPAGE electrophoresis; 2: TRAP-cage with GFP(−21); 3: TRAP-cage with GFP(−21) decorated with Alexa-647; 4: TRAP-cage with GFP(−21) decorated with Alexa-647 and PTD4; 5: GFP(−21)—positive control.

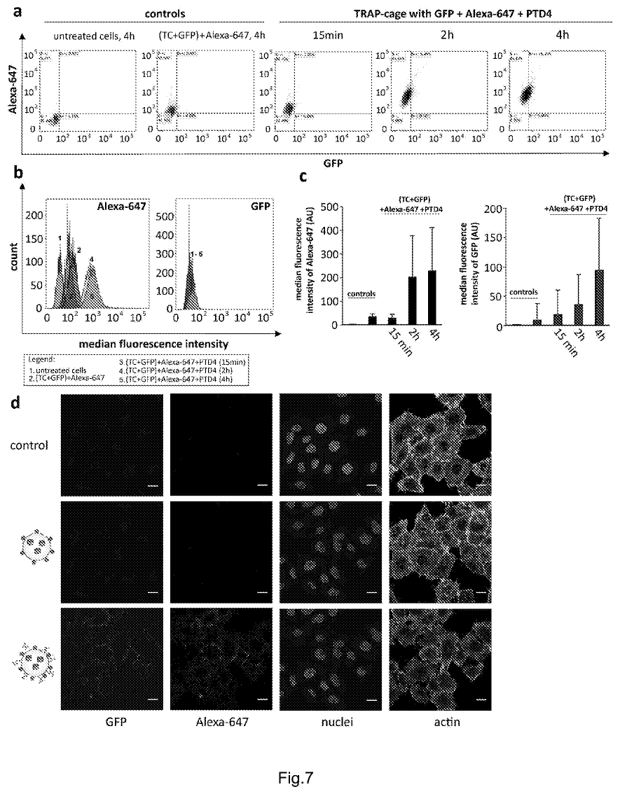

FIG. 7. TRAP-cage stability in culture medium and cell viability test. (a) Native PAGE gels showing TRAP-cage stability in DMEM culture medium without and with FBS presence during 18 h incubation. (b) Cell viability of MCF-7 and HeLa cells after 4 h exposure to empty TRAP-cage, TRAP-cage loaded with GFP(−21) and TRAP-cage with GFP(−-21) decorated with Alexa-647 and PTD4. M =molecular weight marker for native electrophoresis; TC: empty TRAP-cage; (TC+GFP): TRAP-cage filled with GFP(−21); (TC+GFP)+Alexa-647+PTD4: TRAP-cage with GFP(−21) and decorated with Alexa-647 and PTD4.

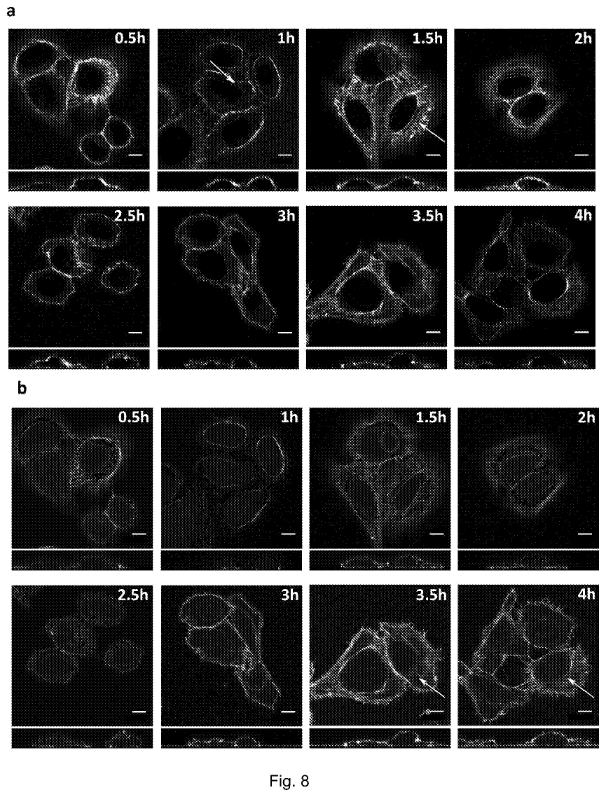

FIG. 8. Delivery of TRAP-cage with GFP(−21) to HeLa cells. (a) Representative flow cytometry dot plots of HeLa cells after treatment with Alexa-647 labeled TRAP-cage with GFP(−21) for 4 h (denoted as (TC+GFP) +Alexa-647) and Alexa-647 labeled TRAP-cage with GFP(−21) and PTD4 (denoted as (TC+GFP) +Alexa-647 +PTD4) for 15 min, 2 h and 4 h. The x-axis and the y-axis show the fluorescent intensities of GFP and Alexa-647 respectively. Untreated cells were used as the negative control. (b) Representative red and green fluorescence overlay histogram plot of the HeLa cells from the same experiment. (c) Median fluorescence intensity of Alexa-647 and GFP positive cells treated with (TC+GFP) +Alexa-647 and (TC+GFP) +Alexa-647 +PTD4 after 15 min, 2 h and 4 h incubation. Data are normalized to untreated cells and based on three independent experiments. Controls: 1: untreated cells; 2: cells incubated with (TC+GFP)+Alexa-647 (d). Confocal microscopy images of untreated cells (control cells) (upper row), cells incubated with (TC+GFP) labeled with Alexa-647 only (middle row), cells incubated with TRAP-cage filled with GFP(−21) and labeled with Alexa-647 and PTD4 (bottom row). Actin filaments were stained with phalloidin conjugated to Alexa-568 and nuclei were stained with DAPI. Green channel—GFP; red channel—Alexa-647; blue channel—DAPI; grey channel—Alexa-568; (scale bar: 10 μM).

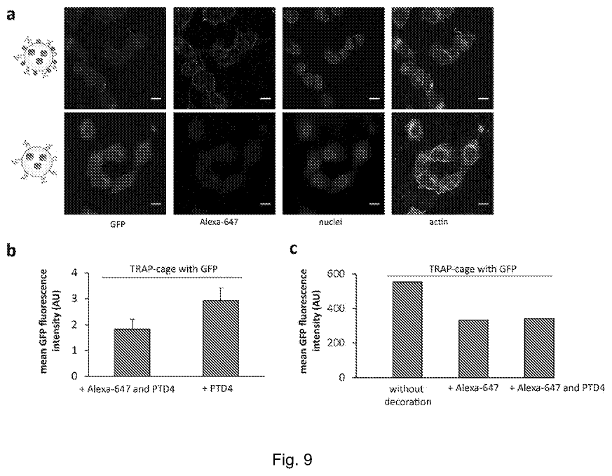

FIG. 9. Tracking TRAP-cage and GFP in HeLa cells. Confocal microscopy merged images of cells incubated with TRAP-cage with GFP(−21) labeled with Alexa-647 and PTD4 and fixed in different time points. Actin was stained with phalloidin conjugated to Alexa-568 whereas DAPI was used for nuclear staining; (scale bar: 10 82 M). Rectangular images are representative orthogonal views in the yz axis. (a)—images with red channel maximal projection; (b)—images with green channel maximal projection.

FIG. 10. Influence of Alexa-647 of GFP(−21) fluorescence. (a) Cells were exposed to (TC+GFP) labeled with Alexa-647 and PTD4 (upper row) or (TC+GFP) labeled with PTD4 only (lower row). Actin filaments were stained with phalloidin conjugated to Alexa-568 and nuclei were stained with DAPI. Green channel—GFP; red channel—Alexa-647; blue channel—DAPI; grey channel—Alexa-568; (scale bar: 10 μM). (b) Mean GFP fluorescence intensity registered from three different fields of view for samples where cells were exposed to (TC+GFP) labeled with Alexa-647 and PTD4 or (TC+GFP) labeled with PTD4 only. The fluorescence intensity was quantified with ImageJ, considering background intensity subtraction. (c) Mean fluorescence of GFP(21) encapsulated in the undecorated and fully decorated TRAP cage, measured in solution.

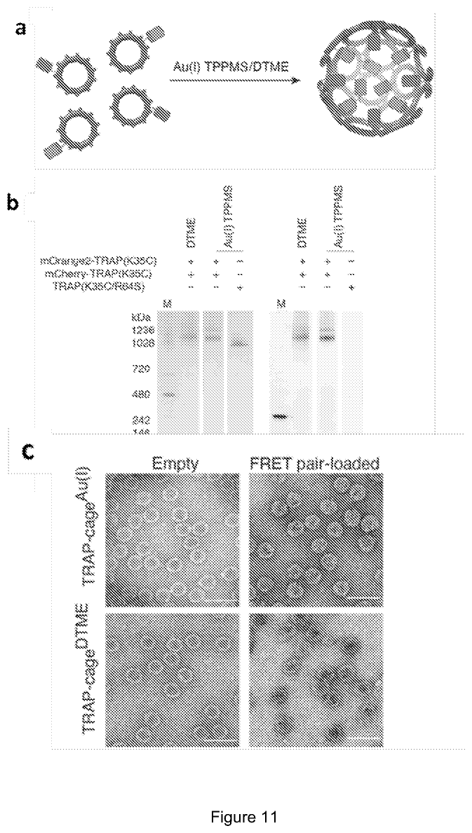

FIG. 11. Guest packaging a single type of guest protein using genetic fusion and patchwork formation. (a) Schematic representation of mCherry encapsulation into TRAP cages using a genetic fusion and patchwork strategy. Ptet/tetO, tetracycline promoter/operon; Pt7/lacO, T7 promoter/lac operon system. (b) Negative-stain transmission electron microscope images of the TRAP cages containing a varied number of mCherry in the lumen.

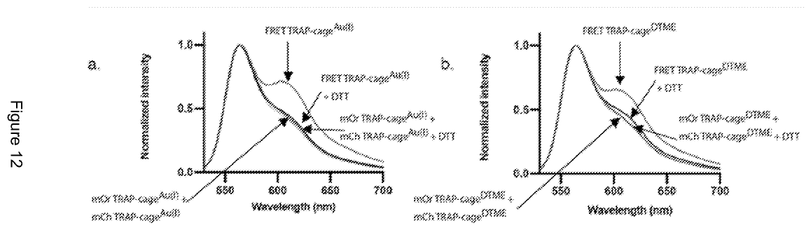

FIG. 12. Guest packaging of two different types of guest protein using genetic fusion and patchwork formation. (a) Schematic representation of TRAP-cage loading with fluorescent proteins. Patchworked TRAP rings fused with either mCherry (dark cylinders) or mOrange2 (light cylinders) at the N terminus were mixed together with either DTME or triphenylphosphine monosulfate (TPPMS)—Au(I)-Cl. (b) Native PAGE showing the fluorescent properties of purified TRAP-cages associated with the fluorescent cargoes. The gel was visualized using InstantBlue protein staining (left) and fluorescence using excitation at 532 nm and emission at 610 nm (right). (c) TEM images of empty (left) TRAP-cages and those filled with fluorescent proteins (right), assembled using either Au(I) (top) or DIME (bottom). Scale bars, 50 nm.

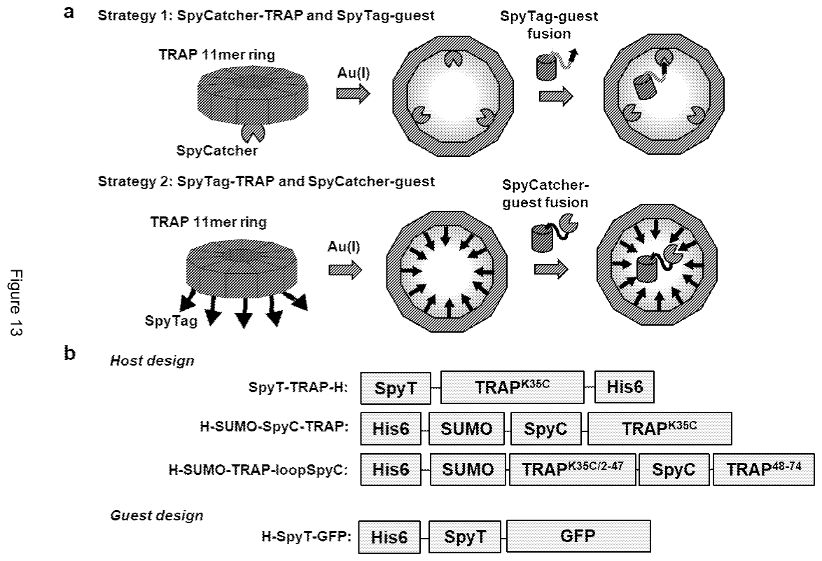

FIG. 13 Confirmation of dual protein loading in TRAP-cage. a, b, Normalized emission spectra of TRAP-cages Au(1) (a) and TRAP-cages lirmE (b) loaded with both mOrange2 and mCherry upon excitation at 510 nm before and after addition of 10 mM DTT. mOrange2 emission peak at 568 nm, mCherry emission peak at 610 nm. Additional lines indicate spectra of cages loaded only with mOrange2 or mCherry proteins mixed together immediately prior to measurement in the absence or presence of DTT, respectively.

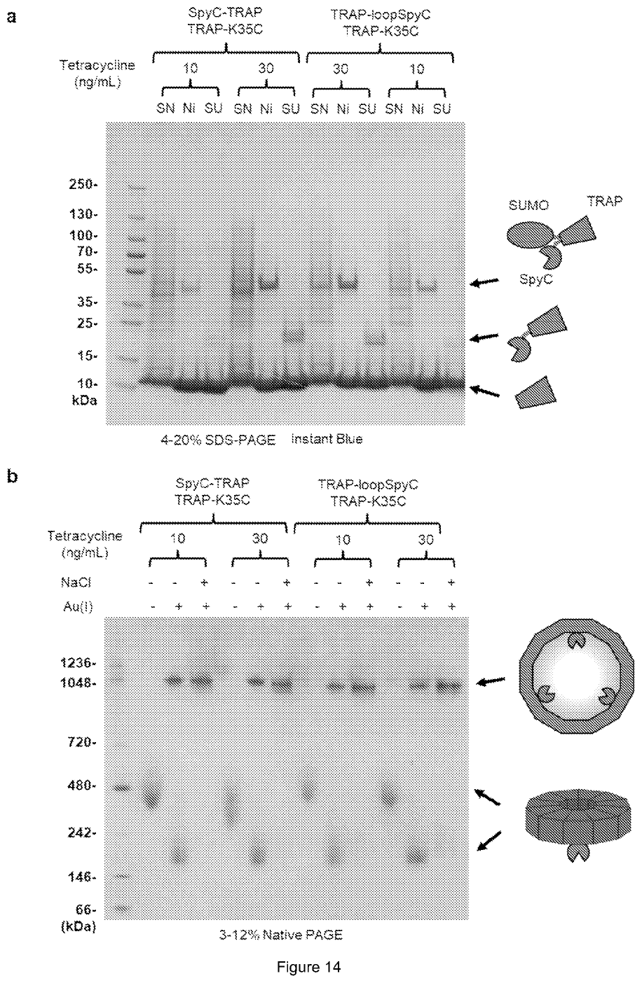

FIG. 14. Concept of the guest packaging using SpyTag/SpyCatcher system. (a) The strategy for SpyTag/SpyCatcher-mediated guest loading. (b) Constructs of TRAP and GFP variants. SpyT, SpyTag; SpyC, SpyCatcher; containing SpyCatcher at the position between residues 47 and 48. This construct was produced with His-tagged SUMO and cleaved by SUMO protease after Ni-NTA affinity chromatography, yielding TRAP-K35C-loopSpyC.

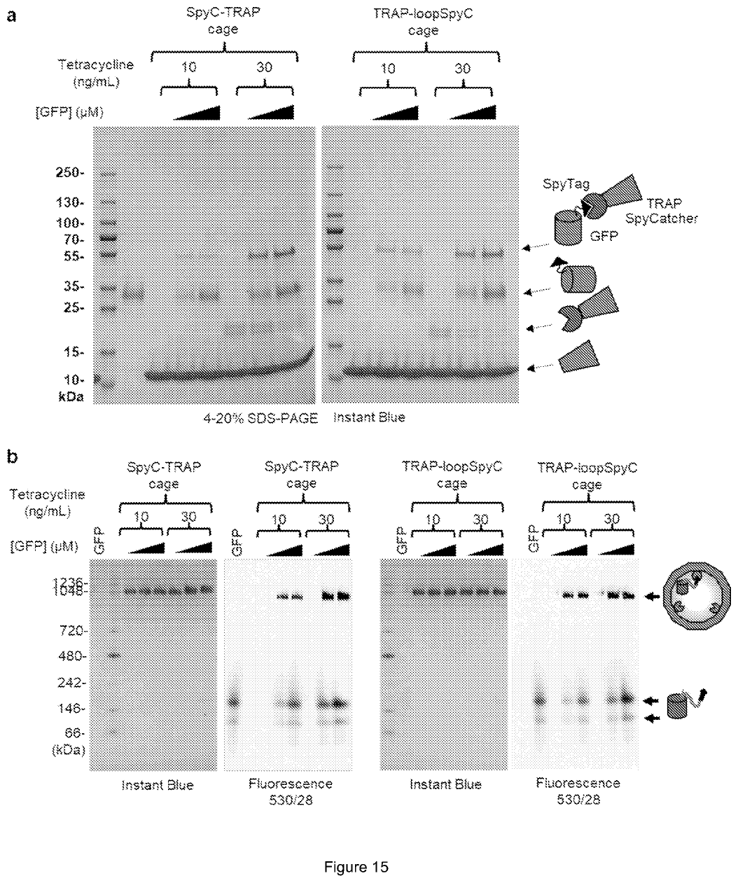

FIG. 15. Production of TRAP cages containing SpyCatcher in the lumen. (a) SDS-PAGE analysis for prodution of TRAP 11 mers composed of TRAP-K35C and either SpyC-TRAP or TRAP-loopSpyC. SN, supernatant after cell lysis and centrifugation; Ni, of Ni-NTA affinity chromatography; SU, SUMO protease cleavage. (b) Native-PAGE analysis for the cage formation with Au(I). The reaction was performed in 50 mM sodium-phosphate buffer (pH 8.0) containing 100 μM TRAP, 0 or 100 μM TPPMS-Au(1)-Cl (Au(I) (−) or (+)), and 0 or 600 mM NaCl (NaCl (−) or (+)).

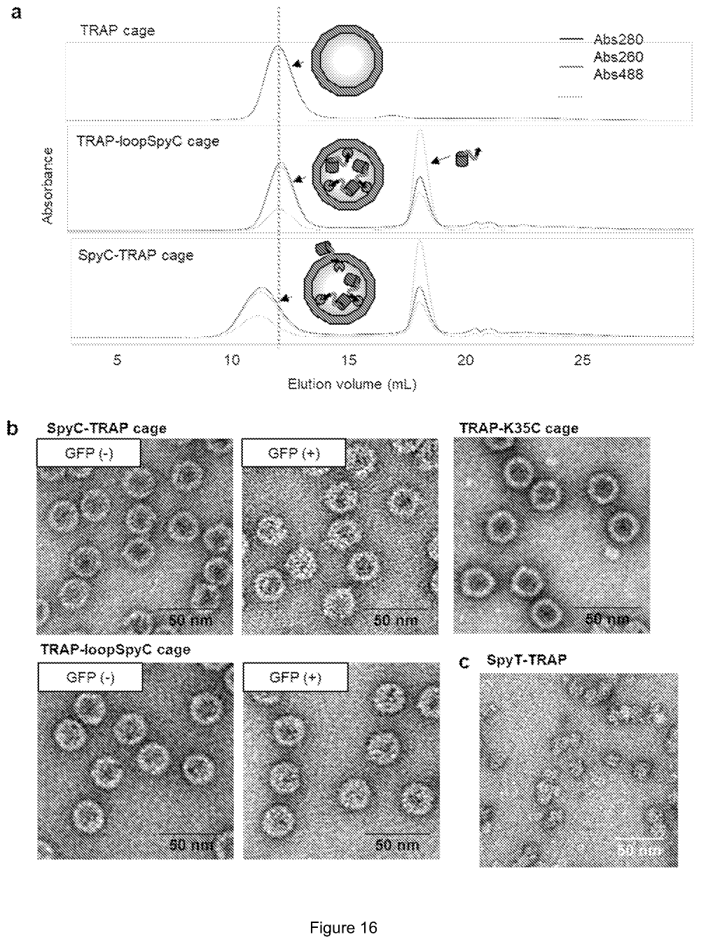

FIG. 16. GFP encapsulation in TRAP cages using SpyTag-SpyCatcher system. (a,b) SDS-(a) and native-(b) PAGE analysis of the mixture of TRAP cages containing SpyCatcher moieties in the lumen, SpyC-TRAP cage or TRAP-loopSpyC cage, and SpyTag-GFP. For the native-PAGE, the same gel was visualized by fluorescence and Instant Blue staining.

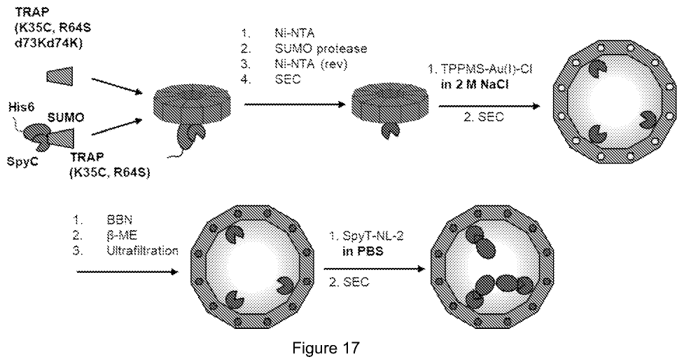

FIG. 17. Isolation and imaging of TRAP cages filled with GFP. (a,b) Size-exclusion chromatogram (a) and negative-stain TEM images (b) of TRAP cages containing SpyCatcher moieties in the lumen, obtained with 30 ng/mL tetracycline induction, mixed with SpyTagged GFP. Of those with cages composed of the TRAP-K35C variant and not filled with any cargo are also provided for a comparison. (c) Negative-stain TEM image of SpyT-TRAP variant.

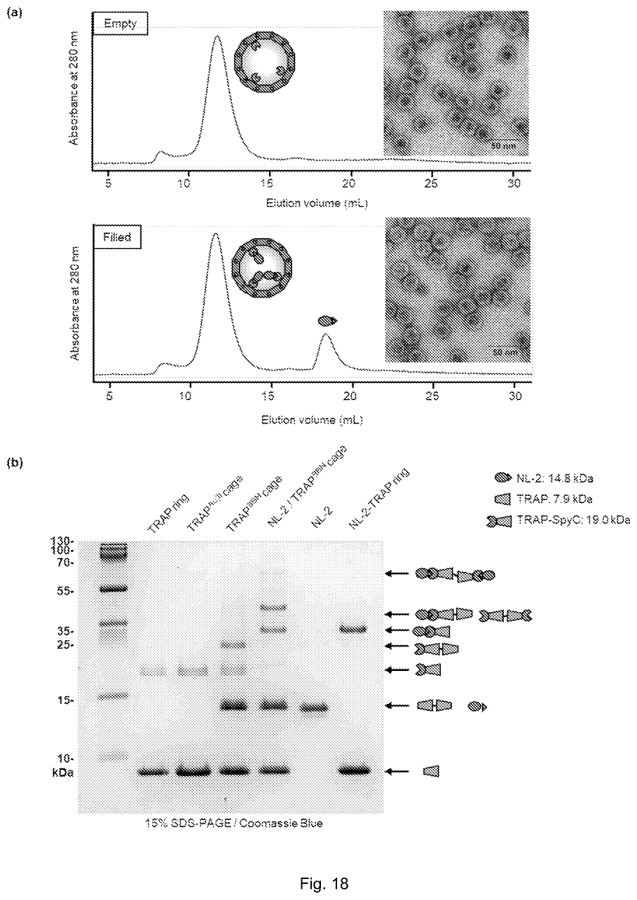

FIG. 18. Strategy for encapsulation of Neoleukin-2/15 in a photo-openable TRAP-cage. The patchwork TRAP ring is composed of a TRAP variant, containing K35C, R64S mutations and lacking lysines at the positions 73 and 74, and the one containing K35C mutation, a His-tag and SUMO at the N-terminus and SpyCatcher in the lumenal loop. BBN, 1,2-bisbromomethyl-3-nitrobenzene; β-ME, β-mercaptoethanol.

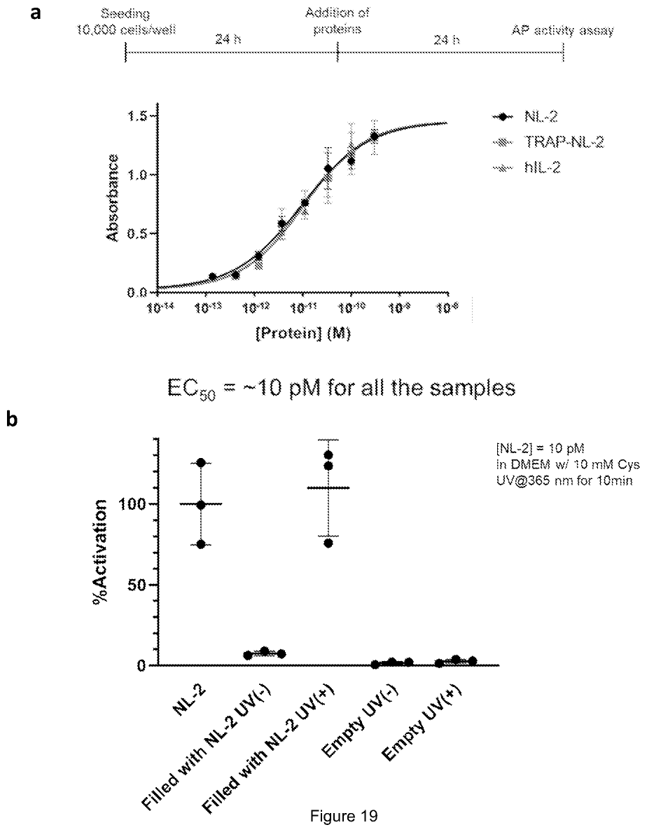

FIG. 19 Triggered release of Neoleukin-2 from TRAP-cage stimulates target cells. a, Graph reflects SEAP activity indicated by absorbance measured after 24 h stimulation of HEK-Blue cells with NL-2, hIL-2, Spy-Tag-NL-2 conjugated with SpyCatcher-TRAP-rings; b, activity of SEAP after 24 h stimulation with NL-2, empty TRAP-cage before and after UV irradiation and SpyCatcher-TRAP-cage filled with SpyTag-NL-2 before and after UV irradiation.

### EXAMPLES

### Techniques Employed in the Realisation of the Invention

### Electron Microscopy

TRAP-cage filled with GFP(−21), TRAP-cage filled with GFP(−21) and labelled with Alexa-647, and TRAP-cage filled with GFP(−21) and fully decorated were imaged using a transmission electron microscope. Samples were typically diluted to a final protein concentration of 0.025 mg/ml, centrifuged at 10,000 g, 5 min, at room temperature and the supernatant applied onto hydrophilized carbon-coated copper grids (STEM Co.). Sample were then negatively stained with 3% phosphotungstic acid, pH 8, and visualized using a JEOL JEM-2100 instrument operated at 80 kV.

### Flow Cytometry

For TRAP-cage internalization experiments, MCF-7 and HeLa cells were seeded into 12-well plates (VWR) in 800 pl of DMEM medium with 10% FBS at a density of 2.5×10⁵ per well and cultured for a further 16 h prior to the experiments. Cells were then incubated with 50 μg (6 nM) of TRAP-cage filled with cargo, labelled with Alexa-647 only or decorated with Alexa-647 and PTD4 peptide in 50 mM HEPES with 150 mM NaCl pH 7.5 supplemented with 10% FBS for 15 min, 2 h and 4 h. After the incubation, cells were washed three times for 5 min with phosphate buffered saline (PBS) (EURx), harvested with trypsin (1 mg/ml) and centrifuged at 150 g for 5 min. Subsequently, cells were washed thrice in PBS by centrifugation (150 g for 3 min) and re-suspended in PBS. Cells were run in Navios flow cytometer (Beckman Coulter) and the fluorescence of 12000 cells was collected per each sample. Untreated cells and cells treated with TRAP-cage filled with cargo and labelled with Alexa-647 only were used as negative controls. Obtained data for three independent experiments were analyzed with Kaluza software (Beckman Coulter). The percentage of Alexa-647/GFP positive cells and median fluorescence intensity was determined for each sample.

### Laser Scanning Confocal Microscopy

For fluorescent laser scanning confocal microscope observations, cells were grown on 15-mm glass cover slips plated into 12-well plates (2.5×10⁵ per well in 800 μl DMEM medium with 10% FBS) and further stimulated as described above for flow cytometry experiments. Next, cells were washed with PBS (3 times for 5 min), fixed with 4% paraformaldehyde solution (15 min, at room temperature) and permeabilized with 0.5% Triton-X100 in PBS (7 min, at room temperature). Actin filaments were stained with phalloidin conjugated to Alexa-568 in PBS (1:300, Thermo Fisher Scientific, 1.5 h, at room temperature). Cover slips were then mounted on slides using Prolong Diamond medium with DAPI (Thermo Fisher Scientific). Fluorescent images were acquired under Axio Observer.Z1 inverted microscope (Carl Zeiss, Jena, Germany), equipped with the LSM 880 confocal module with 63× oil immersion objective. Images were processed using ImageJ 1.47v (National Institute of Health).

### Example 1. Filling of TRAP-cage.

To fill TRAP-cage we took advantage of the fact that the only significant patch of positive charge on the surface of the TRAP ring lies on the face lining the interior of the cage FIG. 1a-c 1b, c). In principle this could allow capture of negatively charged cargoes via electrostatic interaction as has been demonstrated for other protein cages (e.g.⁶) The fact that the constituent TRAP rings do not assemble into TRAP-cage until the addition of gold(I)¹ means that protein cargoes below approximately 4 nm have two possible routes to encapsulation—they may bind to TRAP rings prior to assembly or they may be added after TRAP-cage formation and allowed to diffuse into the cage through the 4-fold holes. We chose negatively supercharged GFP(−21) as a model cargo (FIG. 1d). This cylindrically shaped protein has a diameter of approximately 2.4 nm and is therefore expected to be able to diffuse into the assembled TRAP-cage (FIG. 1e). His-tagged GFP(−21) was mixed with TRAP-cages and incubated overnight, followed by size exclusion chromatography purification for removal of remaining free GFP(−21). It was found that the two proteins associated as shown by co-migration of fluorescence signals on native gels (FIG. 2a). To verify whether His-tagged GFP(−21) is inside the TRAP-cage and not bound to its exterior, we conducted a pull down assay using Ni-NTA affinity chromatography, followed by Western blot analysis. The observation that the GFP(−21) associated with TRAP-cage did not bind to the Ni-NTA column suggested successful encapsulation, making the His-tag inaccessible. This was further supported by a pull down assay which showed that the associated GFP(21) was only available to interact with a Ni-NTA column after the cage was dissociated by the addition of reducing agent (FIG. 2b). These results strongly suggest encapsulation of GFP in TRAP-cage in either full of partial modes (partial encapsulation being the case where the GFP “plugs” the holes in TRAP-cage with the His-tags pointing to the interior). The number of GFP(−21) per cage was approximately 0.3, comparable to that found in a number of other filled protein cages though some have shown considerably greater numbers of cargoes.

### Production and Purification of TRAP-cage Filled With GFP(−21)

TRAP-cage production and purification was performed as described previously.¹ For relevant plasmid and amino acid sequence information see Table 1. Supercharged (21) His-tagged GFP protein was expressed from pET28a encoding the GFP gene and produced in BL21(DE3) cells. The protein was purified using Ni-NTA. Briefly, cells were lysed by sonication at 4° C. in 50 mM Tris-HCl, pH 7.9, 150 mM NaCl, 5 mM MgCl2, 5 mM CaCl₂, in presence of protease inhibitors (Thermo Fisher Scientific), and lysates were centrifuged at 20,000 g for 0.5 h at 4° C. The supernatant was incubated with agarose beads coupled with Ni²⁺-bound nitrilotriacetic acid (His-Pur Ni-NTA, Thermo Fisher Scientific) preequilibrated in 50 mM Tris, pH 7.9, 150 mM NaCl, 20 mM imidazole (Buffer A). After three washes of the resin (with Buffer A) the protein was eluted with 50 mM Tris, pH 7.9, 150 mM NaCl, 300 mM imidazole (Buffer B). Fractions containing His-tagged GFP(−21) were pooled and subjected to size exclusion chromatography on a HiLoad 26/600 Superdex 200 pg column (GE Healthcare) in 50 mM Tris-HCl, pH 7.9, 150 mM NaCl at room temperature. Protein concentrations were measured using a Nanodrop spectrophotometer using a wavelength of 280 nm.

GFP encapsulation was conducted by mixing equal volumes of 100 μM negatively supercharged (−21) His-tagged GFP with 1 μM pre-formed TRAP-cage incubating overnight in 50 mM Tris, 150 mM NaCl, (pH 7.9). Purification of TRAP loaded with GFP was carried out by size exclusion chromatography using a Superose 6 Increase 10/300 column (GE Healthcare) in 50 mM HEPES, pH 7.5, 150 mM NaCl. Fractions containing TRAP-cage were collected and analyzed by native PAGE using 3-12% native Bis-Tris gels (Life Technologies) followed by fluorescence detection using a Chemidoc detector (BioRad) with excitation at 488 nm.

### Estimating the Number of His-tagged GFP(−21) Molecules in the TRAP-cage

Two methods were used for estimating the loading of GFP(−21):
1. Based on detection of GFP fluorescence in TRAP-cage filled with cargo. A GFP(21) standard curve was prepared in the concentration range of 0-100 nM. The fluorescence spectra were acquired at 26° C. using a RF-6000 Shimadzu® Spectro Fluorophotometer with a fixed excitation wavelength at 488 nm and emission wavelength range of 495-550 nm, with an interval of 1.0 nm for Aem, scan speed 6000 nm min, λₑₓ bandwidth 5 nm and λₑₘ bandwidth 5 nm. The fluorescence at emission maximum λₑₘ 510 nm was used for calculation. TRAP protein concentration was determined from absorbance at 280 nm. A TRAP-cage : GFP(−21) stoichiometry of 1:0.28±0.07 was obtained (FIG. 5a).
2. Densitometry analysis. Briefly, a series of His-tagged GFP(−21) dilutions (0.4 ng; 0.8 ng; 4 ng; 8 ng as measured by Nanodrop at wavelength 280 nm) and TRAP-cage filled with cargo, sample (2 μg as measured by Nanodrop at wavelength 280 nm) were separated by SDS-PAGE and subjected to Western blotting (FIG. 5b).The signal from His-tagged GFP(31 21) protein was detected with anti-GFP antibody and secondary HRP-conjugated antibody in a chemiluminescence detector (Chemidoc, BioRad). Densitometry analysis using ImageLab (BioRad) software of the resulting blot showed that 0.6 ng of His-tagged GFP(−21) was present in 2 μg of TRAP-cage filled with cargo. The densitometry analysis yielded a TRAP-cage : GFP(−21) stoichiometry of approx: 1:0.4.

### Ni-NTA “Pull Down”

Samples of purified TRAP-cage filled with His-tagged GFP(−21) protein were divided into two portions and incubated under reducing (1 mM TCEP) or non-reducing (no TCEP) conditions. Next, samples were passed through a Ni-NTA resin (Thermo Fisher

Scientific) under gravitational flow in which 100 μl of each sample was introduced onto 50 μl of the resin equilibrated with Buffer A. Three samples were collected: (i) flow through, (ii) wash with Buffer A and (iii) elution with Buffer B. Samples were analyzed by native PAGE, followed by fluorescence detection (excitation at 488 nm, Chemidoc, BioRad) and Western blot. For the SDS-PAGE and Western blot samples collected from the Ni-NTA pull down assay were denatured by addition of TCEP (final concentration 0.1 mM) and boiling for 15 min followed by separation via Tris/Glycine gel electrophoresis. The gel was subjected to electrotransfer (2 h, 90 V) in 25 mM Tris, 192 mM glycine, 20% methanol buffer onto an activated PVDF membrane. The membrane was blocked with 5% skimmed milk in Tris-buffered saline supplemented with 0.05% of Tween 20 (TBS-T), followed by 1.5 h incubation with mouse monoclonal anti-GFP antibody (1:2500; St. John's Laboratories, UK) and anti-mouse (1:5000, Thermo Fisher Scientific) secondary antibody conjugated with horse radish peroxidase. The signal was developed using a Pierce ECL Blotting Substrate (Thermo Fisher Scientific) and visualized in a BioRad Chemidoc detector.

TABLE 1
Plasmid information and amino acid sequences

| Sequence | Plasmid | | | |
|---|---|---|---|---|
| ID | name | Plasmid | Gene | Amino acid sequence |
| SEQ ID | pET21b_ | pET21b | TRAP- | MYTNSDFVVIKALEDGVNVIG |
| NO: 3 | TRAP-K35C- | | K35C- | LTRGADTRFHHSECLDKGEVL |
| | E48Q-H | | E48Q | IAQFTQHTSAIKVRGKAYIQTR |
| | | | | HGVIESEGKK |
| SEQ ID | pET21b_ | pET21b | TRAP- | MYTNSDFVVIKALEDGVNVIG |
| NO: 4 | TRAP-K35C- | | K35C- | LTRGADTRFHHSECLDKGEVL |
| | E48K-H | | E48K | IAQFTKHTSAIKVRGKAYIQTR |
| | | | | HGVIESEGKK |
| SEQ ID | pET21b_ | pET21b | TRAP- | MYTNSDFVVIKALEDGVNVIG |
| NO: 5 | TRAP-K35C | | K35C | LTRGADTRFHHSECLDKGEVL |
| | | | | IAQFTEHTSAIKVRGKAYIQTR |
| | | | | HGVIESEGKK |
| SEQ ID | pET21b_ | pET21b | TRAP- | MYTNSDFVVIKALEDGVNVIG |
| NO: 6 | TRAP-K35C | | K35C | LTRGADTRFHHSECLDKGEVL |
| | R64S | | R64S | IAQFTEHTSAIKVRGKAYIQTS |
| | | | | HGVIESEGKK |
| SEQ ID | pET28a_GFP | pET28a | GFP | HHHHGSACELMVSKGXELXX |
| NO: 7 | (−21) | | (−21) | GVVPILVELDGDVNGHEFSV |
| | | | | RGEGEGDATEGELTLKFICTT |
| | | | | GKLPVPWPTLVTTLTYGVQCF |
| | | | | SRYPDHMKQHDFFKSAMPEG |
| | | | | YVQERTISFKDDGTYKTRA |
| | | | | EVKFEGDTLVNRIELKGIDFKE |
| | | | | DGNILGHKLEYNFNSHDVYI |
| | | | | TADKQENGIKAEFEIRHNVED |
| | | | | GSVQLADHYQQNTPIGDGPV |
| | | | | LLPDDHYLSTESALSKDPNEK |
| | | | | RDHMVLLEFVTAAGITHGM D |
| | | | | ELYK |
| Sequence | | | | |
| ID | Peptide | | | Amino acid sequence |
| SEQ ID | PTD4 | | | Ac-YARAAARQARAG |
| NO: 9 | | | | |

The TRAP-cages herein may have a a supercharged lumen. In order to have this, the TRAP cage may comprise a E48Q or a E48K mutation. Preferably the TRAP-cage with a supercharged lumen will comprise a K35C/E48Q or a K35C/E48K mutation. This provides and additional

### Example 2. Decoration of TRAP-cage with Fluorescent Dye and With Cell Penetrating Peptide Labelling

We aimed to modify the TRAP-cage in order to promote its cell entry. We choose PTD4 (YARAAARQARA, SEQ. ID No. 8)—an optimised TAT-based cell-penetrating peptide that shows significantly improved ability to penetrate cell membranes, being more amphipathic with a reduced number of arginines and increased α-helical content.⁷ A number of works have shown that coating nanoparticles with PTD4 or similar promotes cell penetration (e.g.⁸). We attached the PTD4 derivative, Ac-YARAAARQARAG (SEQ. ID No. 9), to the amino groups on surface exposed lysines of TRAP-cages. There are three such surface exposed lysines per monomer on TRAP-cage, potentially allowing 792 peptides to be attached per cage. Acetylation of the N-terminal amino group eliminates the possibility of cross-reaction of those amino groups with activated carboxyl moieties that are intended to react with available amino groups of TRAP protein. Additionally, the extended C-terminal glycine residue serves as a flexible linker and as it is not a chiral amino acid, abolishes the chance of racemization during carboxyl activation. The peptide was synthesized using solid-phase methodology and purified by reversephase high-performance liquid chromatography (FIG. 6a). In optimised reactions we observed an increase in the apparent molecular weight of TRAP-cage after reaction with PTD4 (FIG. 6c) as visualised by native PAGE.

In order to be able to track TRAP-cage independently from its cargo we labelled it with Alexa-647 fluorescent dye. For this we cross-linked the maleimide group on the dye with the 24 available cysteines lining the six 4-nm holes of TRAP-cage that are not involved in ring-ring interactions. By titration we established the optimal amount of Alexa-647 (which was equal to the number of TRAP cysteine groups) to be added, where the TRAP-cage is readily labelled and no free dye is present in the sample. This was assessed by native PAGE combined with fluorescent measurements to detect both GFP(−21) and Alexa-647 (FIG. 6b). Although the cargo GFP contains 3 cysteine residues, control reactions showed no detectable labelling of GFP with Alexa647 (FIG. 6c). Negative stain transmission electron microscopy (TEM) confirmed that the modified TRAP-cages retained their characteristic shape (FIG. 6d).

### PTD4 Peptide Synthesis

PTD4 peptide derivative (Ac-YARAAARQARAG, (SEQ. ID No. 10) for simplicity called PTD4 in the text) was synthesized at 0.1 mmol scale using a Liberty Blue automated microwaved synthesizer (CEM, USA), according to the Fmoc-based solid phase peptide synthesis methodology. Fmoc-Gly-Wang resin (100-200 mesh, substitution 0.70 mmol/g, Novabiochem, Germany) was swelled overnight with dichloromethane (DCM)/dimethylformamide (DMF) (1:1). Fmoc-deprotection was performed with 25% morpholine in DMF for 5 min at 85° C. Coupling reactions were performed as per recommended manufacturer's protocol using DIC/oxyma activators with a fivefold excess of Fmoc-protected amino acid derivatives for 5 min at 85° C. Double coupling was applied for all Fmoc-Arg (Pbf) coupling. N-terminal acetylation was performed on resin with 10% acetic anhydride in DMF at 60° C. Cleavage from the resin and side chains deprotection were achieved by treatment with TFA/Triisopropylsilane (TIS)//water (94:3:3) for 4 h with vigorous shaking at 30° C. The resin was filtrated and TFA was evaporated under a mild nitrogen stream. The crude peptide was precipitated by addition of cold diethyl ether, followed by centrifugation (3000 rpm, 10 min). The residue was washed with cold ether (2×) and ethyl acetate (2×). Precipitated crude peptide was dried in vacuo overnight. Crude peptide was dissolved in 8 M urea and purified on an Agilent 1260 RP-HPLC using semi-preparative C18 (10×150 mm) column (Cosmosil, Nacalai tesque). Collected peptide-containing fractions were lyophilized. Purified peptide was analyzed on an analytical C18 column (Zorbax SBC18 5 mm 4.6×150 mm, Agilent) in a linear gradient of 0-20% of acetonitrile with 0.1% TFA for 30 min at flow rate 1.0 ml/min. Peak signals were detected at 220 and 280 nm (FIG. 6a).

### TRAP-cage Labeling with Alexa-647 and Decoration with Cell-penetrating Peptide

Alexa Fluor-647 C2 maleimide fluorescent dye (Alexa-647, Thermo Fisher Scientific) and cell-penetrating PTD4 peptide were conjugated to the TRAP-cage filled with GFP via a crosslinking reactions with cysteines and lysines present in the TRAP protein.

To achieve fluorescent labelling, TRAP-cage carrying GFP (300 μl, 16 nM) was mixed with a Alexa-647 C2 maleimide dye (50 μl, 1 μM), the reaction was conducted in 50 mM HEPES with 150 mM NaCl pH 7.5 for 2.5 h at room temperature with continuous stirring at 450 rpm. The optimal interaction ratio of maleimide-conjugated Alexa-647 to TRAP-cage was assessed by titration (FIG. 6b). Briefly, aliquots of TRAP-cage loaded with GFP(−21) (11.36 nM) were mixed with maleimide-conjugated Alexa-647 ranging from 0.1 μM to 100 μM. Samples were then separated by native gel electrophoresis and visualized by fluorescence detection in a Chemidoc, with excitation at 647 nm. Reactions where no free Alexa-647 is present in the sample and no GFP interference with the Alexa-647 signal is observed, were considered as optimal decoration conditions and used in further experiments.

Additionally, to rule out a possibility of direct GFP labeling by Alexa-647, TRAP-cage loaded with GFP(−21) with and without Alexa-647 labelling were subjected to denaturing gel separation and Western blotting followed by detection with anti-GFP antibody. No band shift from potential interaction of GFP with Alexa-647 dye was observed (FIG. 6c).

For the cell-penetrating peptide decoration, PTD4 peptide (50 μl, 0.5 mM) was mixed with 1-ethyl-3-(−3-dimethylaminopropyl) carbodiimide hydrochloride (EDC, 10 μl, 83 mM) and N-hydroxysuccinimide (NHS, 10 μl, 435 mM), all reagents dissolved in ddH₂O. Subsequently, the excess of activated PTD4 peptides were added to TRAPcage filled with GFP(−21) and labelled with Alexa-647 and incubated for next 2.5 h at room temperature, with continuous stirring at 450 rpm. The reaction was stopped by addition of 5 μl of 200 mM Tris-HCl pH 7.5. The conjugation efficiency was verified by native PAGE and fluorescent gel imaging. A change in molar weight of the decorated TRAP-cage results in a band shift observed in native PAGE (FIG. 6c).

### Example 3. Stability of TRAP-cage and Effect on Cell Viability

Before embarking on cell delivery tests, we firstly assessed whether TRAP-cage was structurally stable, i.e. did not disassemble under cell culture conditions. Stability was checked at 37° C., 5% CO₂ atmosphere in Dulbecco's Modified Eagle Medium (DMEM) without or with foetal bovine serum (FBS) at various concentrations. The results showed that the cage structure is stable in the DMEM culture medium within 18 h incubation at 37° C., 5% CO₂ (FIG. 7a).

In order to determine the effect of TRAP-cage on cell viability alamarBlue assays were carried out. This test is based on the natural ability of viable cells to convert resazurin, a blue and nonfluorescent compound, into resofurin; a red and fluorescent molecule by mitochondrial and other reducing enzymes.⁹ Human cancer cell lines MCF-7 and HeLa were incubated in the presence of a TRAP-cage, TRAP-cage filled with GFP(21) and decorated with Alexa-647 and PTD4 peptide. The number of cells, TRAP-cage dose and stimulation time used in cell viability tests correspond to the conditions under which the internalization of the TRAP-cage experiments were performed. Untreated cells were used as a control. The data showed that both unmodified TRAP-cage and TRAP-cage filled with GFP(−21) and decorated with Alexa-647 and PTD4 do not significantly affect the viability of MCF-7 and HeLa cells for at least 4 h of incubation (FIG. 7b).

### Cell Culture and Cytotoxicity Assessment of the TRAP-cage

HeLa and MCF-7 cells were cultured in Dulbecco's Modified Eagle Medium (DMEM, Sigma) supplemented with 10% FBS (EURx), 100 μg/ml streptomycin, 100 IU/ml penicillin (Gibco). The culture was maintained at 37° C. under 5% CO₂. To test TRAP-cage stability in the culture medium, purified sample was added to DMEM medium containing 0, 2 and 10% fetal bovine serum (FBS) and incubated at 37° C. under 5% CO₂ for 2 h, 6 h and 18 h. Samples were subsequently analyzed by native PAGE followed by Instant blue gel staining (FIG. 7a).

Cell viability after TRAP-cage treatment was determined using the alamarBlue test (VWR). Cells were cultured in 96-well plates at a density of 2.5×10⁴ cells per well. Next, cells were treated with 5 μg (0.6 nM) TRAP-cage, TRAP-cage filled with GFP(21) and decorated with Alexa-647 and PTD4 in 50 mM HEPES with 150 mM NaCl pH 7.5 supplemented with 10% FBS for 4 h. After the treatment, 10 μl of alamarBlue diluted in 90 μl DMEM medium was added per well, and cells were incubated for the next 3 h at 37° C. under 5% CO2. Resazurin, the active component of alamarBlue, was reduced to the highly fluorescent compound resorufin only in viable cells and absorbance (excitation 570 nm, emission 630 nm) of this dye was recorded. Nontreated cells were used as a negative control (FIG. 7b). All samples were measured in triplicates, in three independent experiments.

### Example 4. Delivery of Protein Cargo to Cells

Delivery of TRAP-cage to cells was studied using human cancer cell lines MCF-7 and HeLa. Cells were incubated for different time periods with the purified TRAP-cages containing encapsulated GFP(−21) and labelled with Alexa-647 only or with Alexa-647 and PTD4 and analysed by flow cytometry. The fluorescent signal due to both Alexa647 and GFP increased with prolonged incubation time in both cell lines treated with TRAP-cage with GFP labelled with Alexa-647 and PTD4 peptide (FIG. 3a, b, c). These results show that external modification of TRAP-cages with cell penetrating peptides promote their cell entry and effective cargo delivery. Interestingly, this effect was more pronounced in the case of the MCF-7 cell line compared to the HeLa cell line (FIG. 8a, b, c).

In order to discriminate between fluorescent signals from TRAP-cages which were internalized in the cells and those which were adsorbed externally on the cell membrane, confocal microscopy was used. TRAP-cage containing GFP(−21) and labelled with Alexa-647 but lacking PTD4 were not observed in the cells. In contrast, TRAP-cage containing GFP(−21) and decorated with PTD4 showed a clear signal in the cell interior 4 h after stimulation (FIG. 3d and FIG. 8d).

### Example 5. Intracellular Dynamics of TRAP-cage

The high stability of TRAP-cage coupled with its ability to break apart in presence of modest concentrations of cellular reducing agents suggests that TRAP-cage in the cytoplasm should readily disassemble, releasing GFP(−21) cargo. As TRAP-cage and GFP possess discrete and trackable signals we hypothesized that cage disassembly and release of GFP(−21) may be strongly inferred if the Alexa-647 and GFP signals became non-colocalised after cell entry. To assess this possibility, we tracked both signals over time after addition to MCF-7 and HeLa cancer cells. Notably, in both cell lines tested, during the first 90 minutes of incubation, TRAP-cage was mainly present at the cell boundaries as indicated by the strong localisation of the Alexa-647 signal there (FIG. 4a, 9a) and the GFP signal was barely detectable (FIG. 4b, 9b). However, after 3 h of incubation, the TRAP-cage signal (Alexa-647) became weaker and appeared to be distributed more evenly in the cell, whereas the GFP signal was clearly detectable, due likely to its release from the TRAP-cages (FIG. 4a, b and FIG. 9a, b).

### Example 6. Influence of Alexa-647 of GFP(−21) Fluorescence

To assess the potential influence of Alexa-647 on GFP(−21) fluorescence (suggested by FIG. 6a, middle panel) we compared, by confocal microscope imaging, TRAP-cages filled with cargo where the cages compared were either decorated with PTD4 peptide only, or were fully decorated (PTD4 and Alexa-647) (FIG. 10a). Briefly, cells were treated with the respective samples as described in Materials and Methods. Next, cells were fixed and stained following the protocol described above. The fluorescence intensity in the green channel was quantified with ImageJ. Calculations of the mean fluorescence intensity (FIG. 10b) took into account the background signal from each field of view.

Additionally, in-solution fluorescence of GFP(−21) encapsulated in the fully decorated TRAP-cage was compared to the fluorescence of the cargo in the TRAP-cage without Alexa-647 using a RF-6000 Shimadzu® Spectro Fluorophotometer. As shown in FIG. 10c presence of the Alexa-647 dye on the TRAP-cage results in approximately 30% reduction in the fluorescence of its cargo.

### Example 7. Filling TRAP-cage with a Protein Cargo Via Genetic Fusion

Efficient protein packaging was achieved by genetic fusion of guest to the cage-forming TRAP. As an initial model, we employed a far-red fluorescent protein, mCherry (FIG. 11a). The N-terminally His-tagged fluorescent protein was genetically fused to the TRAP^{K35C} N-terminus which faces to the interior when assembled. Since too much modification with these fluorescent proteins to one 11-mer TRAP units might hamper the cage assembly due to the steric hindrance, the fusion proteins were co-produced with unmodified TRAP^{K35C} in the Escherichia coli host cells where the individual transcription level can be controlled by different inducers, tetracycline and isopropyl-β-_{D}-thiogalactoside (IPTG), respectively. Using this co-production system, the number of mCherry per TRAP ring can be well-regulated by altering concentration of tetracycline . The patchwork TRAP rings fused to mCherry were then assembled into cages using Au(I) (FIG. 11b). The same approach can be used to fill the TRAP-cage with more than one type of cargo protein.

### Protein Production

To produce patchwork TRAP rings were co transformed with pACTet_H-mCherry-TRAP-K35C and pET21_TRAP-K35C. Protein expression was induced by addition of 0.2 mM isopropyl-β-d-thiogalactopyranoside and tetracycline (8 ng/ml). After cell lysis by sonication patchwork TRAP rings were then isolated using Ni-nitrilotriacetic acid (NTA) affinity chromatography, followed by SEC using a Superdex 200 Increase 10/300 GL column.

### Cage Assembly and Characterization

Formation of TRAP-cage was carried out by mixing equimolar amounts purified TRAP ring containing mCherry and chloro(triphenylphosphine monosulfoxide)gold(I)-(TPPMS-Au(I)-CI) in 50 mM sodium phosphate buffer (pH 8.0) containing 600 mM NaCl and kept at room temperature overnight. The guest protein stoichiometry was determined using absorbance ratio 280/587 nm. The morphology of the isolated cage was examined using negative stain TEM with the protocol described above.

### TRAP-cage With mCherry Decoration with Cell-penetrating Peptides

A maleimide moiety was introduced at the N-terminus of the peptide on resin using 6-maleimide hexanoic acid and a DIC/Oxyma coupling protocol. The 0.5 mM 6-maleimidehexanoic-PTD4 or HA/E2 (25 μl, 0.5 mM) peptides was mixed with TRAP-cage filled with mCherry (75 μl, 0.3 mg/ml) and incubated overnight at room temperature, with continuous stirring at 450 rpm. The conjugation efficiency was verified by native PAGE and fluorescent gel imaging. A change in molar weight of the decorated TRAP-cage results in a band shift observed in native PAGE.

### Example 9. Filling TRAP-cage With Two Different Protein Cargoes via Genetic Fusion

It is possible to fill TRAP-cage with more than one type of protein. Two fluorescent proteins, mOrange2 and mCherry serving as a Forster resonance energy transfer (FRET) donor and acceptor respectively were encapsulated via the genetic fusion of each cargo protein to the N-terminus of TRAP monomer. The fusion proteins were co-produced with unmodified TRAPK35C in the Escherichia coli host cells where the individual transcription level can be controlled by different inducers, tetracycline and isopropyl-β-D-thiogalactoside (IPTG). The amount of expression inducer added was optimized to obtain 0.3 mOrange2 and 1 mCherry proteins per TRAP-ring which enabled avoiding steric hinderance during a cage formation process.

Cargo modified TRAP-rings were then mixed in 1:1 molar ratio and added with either Au(I)- or DTME to promote cage assembly (FIG. 12a).

Resultant cages were then purified by size-exclusion chromatography and analyzed by native PAGE combined with fluorescence detection and TEM imaging (FIG. 12b,c). Native PAGE confirmed the successful encapsulation of TRAP-cages with both fluorescent cargoes which could be seen by fluorescence excitation at 532 nm (emission 610 nm) cage (FIG. 12b).

TEM imaging showed monodisperse population of TRAP-cages which were clearly packaged with cargo after its assembly with the mixture of cargo-modified TRAP-rings. The resultant retained their morphology as compared to empty Au(I) or DTME induced cages (FIG. 12c).

Presence of mOrange2 and mCherry proteins in the close proximity of TRAP-cages should allow for Förster Resonance Energy Transfer (FRET) which is a physical process where energy is transferred from an excited fluorophore to another molecule. Energy transfer between fluorescent proteins encapsulated inside the protein cages has been already described but has never been applied for the monitoring of disassembly kinetics of artificial protein cages.

To assess the efficiency of FRET between mOrange2 and mCherry proteins inside TRAP-cages^{DTME} and TRAP-cages^{Au(I)} spectral data were gathered using the excitation value for mOrange2. All the spectral data were normalized at mOrange2 fluorescence peak and the co-localization with mCherry was judged by the relative values of fluorescence intensity ratios. Fluorescence spectra were measured not only for FRET pair-packaged TRAP-cages but also for control samples which were TRAP-cages^{Au(I)/DTME} encapsulated only with either mCherry or mOrange2 proteins and mixed afterwards in solution. Such control samples cannot show FRET as the fluorophores are far from each other being encapsulated in distant cages. Indeed, spectra of TRAP-Cage^{Au(I)} and TRAP-cage^{DTME} packaged with the FRET pair showed an approximately 1.5-fold higher signal in mCherry emission at 610 nm, compared to the corresponding control samples (FIG. 13 a,b) which indicates the efficient energy transfer between mOrange2 and mCherry inside both DTME and Au(I) induced TRAP-cages.

### Protein Production

E. coli strain BL21(DE3) cells were co-transformed with either pACTet_H-mOrange-TRAPK35C or pACTet_H-mCherry-TRAP-K35C and pET21_TRAP-K35C. Cells were grown in 100 ml LB medium supplemented with ampicillin and chloramphenicol at 37° C. until OD₆₀₀=0.5-0.7. At this point, protein expression was induced by addition of 0.2 mM IPTG and 10 ng/ml of tetracycline in the case of pACTet_H-mCherry-TRAP-K35C or 30 ng/ml of tetracycline in the case of pACTet_H-mOrange-TRAP-K35C, followed by incubation for 20 hours at 25° C. Cells were then harvested by centrifugation for 10 min at 5,000×g. Cell pellets were stored at −80° C. until purification. Pellets were resuspended in 40 ml lysis buffer (50 mM sodium phosphate buffer, 600 mM NaCl, 10 mM imidazole, pH 7.4) supplemented with DNase I and lysozyme, 1 tablet of protease inhibitor cocktail and 2 mM DTT and stirred for 30 min at room temperature. Then, the samples were sonicated and clarified by centrifugation at 10,000×g, 4° C. for 20 min. The supernatant was then incubated with 4 ml Ni-NTA resin previously equilibrated in lysis buffer in a gravity flow column for 20 min. The resin was then washed more than 10 column volumes in lysis buffer containing 20 and 40 mM imidazole. His-tagged proteins were eluted using 5 ml of 50 mM sodium phosphate buffer containing 500 mM imidazole (pH 7.4). Protein samples were then buffer exchanged using Amicon Ultra-15 centrifugal filter unit (50k molecular weight cut-off (MWCO), Merck Millipore) into 2× phosphate buffered saline (PBS) plus 5 mM ethylenediaminetetraacetic acid (EDTA), referred to as 2×PBS-E herein after. The proteins were then subjected to size-exclusion chromatography using a Superdex 200 Increase 10/300 GL column (GE Healthcare) at 0.8 ml/min flow rate. The main peak showing absorption at 548 nm or 587 nm was pooled and concentrated using ab Amicon Ultra-15 (50k MWCO). Protein purity was checked by SDS-PAGE and protein concentration was determined by absorbance measured using UV-1900 UV-Vis Spectrophotometer (Shimadzu) using extinction coefficients: ϵ_{mCherry 587}=72,000 M⁻¹ cm⁻¹, ϵ_{mOrange 548}=58,000 M⁻¹ cm⁻¹, ϵ_{TRAP 280}=8250 M⁻¹ cm⁻¹ (http://expasy.org/tools/protparam.html). Proteins were stored at 4° C. until use.

### Cages Assembly and Purification

For cross-linker-induced cage assembly, TRAP(K35C/R64S) (100-500 μM) in 2×PBS-E was mixed with 5-fold molar excess of either DTME or BMH and stirred at room temperature for 1 hour. Final DMSO concentration in solution was kept at no greater than 12.5%. After the reaction, the insoluble fraction, likely due to low solubility of cross-linkers in aqueous solution, was removed by centrifugation for 5 min at 12,000×g. Supernatants were then purified by size-exclusion chromatography using a Superose 6 Increase 10/300 GL column (GE Healthcare) at a flow rate of 0.5 ml/min on an ÄKTA purifier FPLC (GE Healthcare). Fractions containing cross-linked TRAP cages were pooled and concentrated using Amicon Ultra-4 (100k MWCO) centrifugal filter units. Typical yield of obtained cross-linked TRAP-cages was approx. 20%. Formation and purification of gold (I)-induced TRAP-cages were performed as previously described (1). Cage formation with fusion proteins were performed using the same protocols as described for both cross-linked and gold (I)-induced cages with an additional Ni-NTA purification step prior to size-exclusion chromatography to purify the sample away from partially assembled cages (His-tagged mCherry and mOrange2 that are not fully protected inside the cages bind to Ni-NTA column). The protein concentration and ratio of encapsulated guests were estimated using the absorbance ratio at 280/548 nm or 280/587 nm using an analogous method to the one previously reported (4). Extinction coefficients used for calculations were ϵ_{mCherry 587}=72000 M⁻¹ cm⁻¹, ϵ_{mOrange2 548}=58000 M⁻¹ cm⁻¹, ϵ_{TRAP 280}=8250 M⁻¹ cm⁻¹. Due to spectral overlap between mCherry and mOrange2, to properly calculate the concentrations of both encapsulated guests, mCherry extinction coefficients was also estimated at 548 nm (ϵ_{mCherry 548}=42538 M⁻¹ cm⁻¹) using the absorbance ratio at 548/587 nm of mCherry without fusion to TRAP. Likewise, the extinction coefficients of mCherry and mOrange2 at 280 nm were experimentally determined as ϵ_{mCherry 280}=56744 M⁻¹ cm⁻¹ and ϵ_{mOrange2 280}=52200 M⁻¹ cm⁻¹ respectively. The morphological fidelity of assembled cages was confirmed by negative stain TEM and native PAGE analysis.

Fluorescence measurements: Fluorescent spectra were acquired at room temperature using 70 nM mOrange2 in 2×PBS-E in a 1-cm-light-pass-length polystyrene cuvette on an RF-6000 Fluorescence Spectrofluorometer (Shimadzu). The proteins were excited at 510 nm and emissions were scanned over a wavelength range from 530 to 700 nm. Obtained spectra were normalized to the mOrange2 fluorescence peak. After each measurement 10 mM DTT was added to the samples to trigger complete cages disassembly.

TABLE 2
Plasmid information and amino acid sequences

| Sequence | Plasmid | | | |
|---|---|---|---|---|
| ID | name | Plasmid | Gene | Amino acid sequence |
| SEQ ID | PACTet_H- | PACYC | His6- | MHHHHHHGGSSMVS |
| NO: 10 | mCherry- | | mCherry- | KGEEDNMAIIKEFMRF |
| | TRAP-K35C | | TRAP- | KVHMEGSVNGHEFEIE |
| | | | K35C | GEGEGRPYEGTQTAK |
| | | | | LKVTKGGPLPFAWDIL |
| | | | | SPQFMYGSKAYVKHPA |
| | | | | DIPDYLKLSFPEGFKWE |
| | | | | RVMNFEDGGVVTVTQD |
| | | | | SSLQDGEFIYKVKLRGT |
| | | | | NFPSDGPVMQKKTMGW |
| | | | | EASSERMYPEDGALKGE |
| | | | | IKQRLKLKDGGHYDAEV |
| | | | | KTTYKAKKPVQLPGAYN |
| | | | | VNIKLDITSHNEDYTIVEQ |
| | | | | YERAEGRHSTGGMDELY |
| | | | | KLSENLYFQSGGSGSSYT |
| | | | | NSDFVVIKALEDGVNVIGL |
| | | | | TRGADTRFHHSECLDKGE |
| | | | | VLIAQFTEHTSAIKVRGKA |
| | | | | YIQTRHGVIESEGKK |
| SEQ ID | pACTet_H- | PACYC | His6- | MHHHHHHGGSSMVSKG |
| NO: 11 | mOrange- | | mOrange- | EENNMAIIKEFMRFKVRM |
| | TRAPK35C | | TRAP- | EGSVNGHEFEIEGEGEG |
| | | | K35C | RPYEGFQTAKLKVTKGG |
| | | | | PLPFAWDILSPHFTYGSK |
| | | | | AYVKHPADIPDYFKLSFPE |
| | | | | GFKWERVMNYEDGGVVT |
| | | | | VTQDSSLQDGEFIYKVKLR |
| | | | | GTNFPSDGPVMQKKTMG |
| | | | | WEASSERMYPEDGALKG |
| | | | | KIKMRLKLKDGGHYTSEV |
| | | | | KTTYKAKKPVQLPGAYIVD |
| | | | | IKLDITSHNEDYTIVEQYER |
| | | | | AEGRHSTGGMDELYKLSE |
| | | | | NLYFQSGGSGSSYTNSDF |
| | | | | VVIKALEDGVNVIGLTRGAD |
| | | | | TRFHHSECLDKGEVLIAQFT |
| | | | | EHTSAIKVRGKAYIQTRHG |
| | | | | VIESEGKK |

### Example 10. Filling TRAP-cage With a Protein Cargo via Isopeptide Bond Formation

Despite the robust and general system using genetic fusion, this strategy still holds a drawback in the requirement to expose guest proteins to Au(I) or maleimide crosslinker. This procedure may particularly be problematic if the guest proteins contain a free cysteine residue that is important for the activity, e.g. cysteine proteases. To overcome the issue, we devised a post-assembly loading system using SpyTag/SpyCatcher system, the 13-amino-acid peptide SpyTag interacts with the protein SpyCatcher to form an isopeptide bond spontaneously (Zakeri B, et al. Proc. Natl. Acad. Sci. U.S.A. 2012, 109, 690-697, which is hereby incorporated by reference). In the context of filling a protein cage, two strategies can be used (FIG. 14a). In strategy 1, the internal wall of TRAP cage bears the SpyCatcher while the cargo carries SpyTag. In the second strategy, the internal wall of the TRAP cage bears a SpyTag while the cargo carries SpyCatcher, We designed various constructs based on this approach firstly for capture of GFP in TRAP-cage (FIG. 14b). For the strategy 2, SpyCatcher was introduced into either the N-terminus or a lumen facing loop of TRAP (specifically between residues 47 and 48). All the host TRAPs were equipped with a hexahistidine tag to facilitate purification, while the tag can be cleaved off using either carboxypeptidase or SUMO protease, to yield TRAP-K35C variants possessing a SpyTag or a SpyCatcher at the N-terminus, referred to as SpyT-TRAP or SpyC-TRAP, respectively, as well as the one having a SpyCatcher in the lumenal loop, referred to as TRAP-loopSpyC.

The TRAP variants possessing a SpyCatcher was coproduced in host bacteria with untagged TRAP-K35C to yield patchwork rings as described in Example 9. To avoid insufficient cage formation due to too much SpyCatcher moieties, we tested two concentrations, 10 or 30 ng/mL, of tetracycline that regulates gene expression level of the SpyCatcher-fusion variants. Production of the patchwork rings with varied contents of the fusions as well as cleavage of the His-tag by SUMO protease were confirmed by SDS-PAGE analysis (FIG. 15a). Upon the addition of Au(I) in a phosphate buffer, all the variants showed cage formation, while the assembly efficiency was further enhanced by increasing the ionic strength of the buffer (FIG. 15b). Following isolation of the assembled cages, they were mixed in phosphate buffered saline (PBS) with a varied concentration of GFP equipped with a SpyTag (SpyTag-GFP). Isopeptide formation as well as the host-guest association were observed for all the variants, judged by analysis using SDS-PAGE (FIG. 16a) and native-PAGE (FIG. 16b), indicating successful guest packaging. However, further analysis of the SpyC-TRAP-cage samples obtained from 30 ng/mL tetracycline using SEC and TEM showed a slight shift in the elution time compared to that of empty TRAP-cage and some objects on the exterior of the cage assembly, suggesting that the guest GFP might be partially leaked from the cages (FIG. 17). Meanwhile, such a leakage was not observed for the TRAP-loopSpyC cages. UV-Vis absorbance of the isolated, filled cages revealed that these TRAP cages can be loaded with upto ˜28 GFP molecules while the packaging density can be controlled by changing either tetracycline concentration in the bacterial production or mixing ratio in the encapsulation process. In contrast to the TRAP-SpyCatcher fusions, the strategy 1 was found to be challenging as the SpyT-TRAP variants showed an aggregation tendency and thus efficient Au(I)-mediated cage formation was not observed (FIG. 17c). Taken all together, it can be concluded that the TRAP-loopSpyC variant is the most robust and efficient variant for guest packaging.

The second guest demonstrated in this way was SpyTagged Neoleukin-2/15 (Silva DA, et al. Nature, 2019, 565, 186-191, which is hereby incorporated by reference), which was also successfully loaded into the TRAP cages possessing SpyCatchers in the lumen. A photo-openable TRAP, composed of the variant containing two mutations, K35C and R64S, and lacking the C-terminal two lysine residues, d73K and d74K and TRAP-loopSpyC, in which the TRAP rings were connected each other with a photocleavable crosslinker, 1,2-bisbromomethyl-3-nitrobenzene (BBN) (FIG. 18). Similar to the GFP case, SpyTagged NL-2 was mixed with the TRAP cage and the filled cages were isolated by size-exclusion chromatography (FIG. 19a). Subsequent analysis using negative-stain TEM and SDS-PAGE revealed that the NL-2 proteins were successfully packaged in the TRAP cages via isopeptide bond formation (FIG. 19b).

HEK-Blue IL-2 cells assay was used to assess the properties of the encapsulated SpyTag-NL-2 in the TRAP-cages. HEK-Blue are the type of HEK 293T cells which were engineered to stably co-express human IL-2 receptor together with its signaling pathway with additional secreted embryonic alkaline phosphatase (SEAP) reporter gene. Binding of IL-2 or NL-2 to the IL-2 receptor (IL-2R) leads to the initiation of the signaling cascade which results in the transcription activation and secretion of SEAP allowing its monitoring by colorimetric method.

HEK-Blue cells were seeded on 96-well plates. The next day encapsulated with NL-2/15 and empty UV-photocleavable SpyCatcher-TRAP-cage samples were added with 10 mM cysteine (quencher) and treated with UV light for 10 min. Treated samples and the controls were diluted in a cellular medium (DMEM) to various concentrations in the pM range. Control samples included unconjugated SpyTag-NL-2 and purchased human IL-2, SpyTag-NL-2 conjugated with TRAP-rings and empty TRAP-cages before and after UV treatment. Cells were stimulated for 24 hours followed by performing Quanti Blue assay which enabled assessing the amount of the secreted SEAP.

HEK-Blue cells treated with SpyTag-NL-2, hIL-2 and TRAP-NL-2 control samples showed a very similar level of produced SEAP after the stimulation which suggests that IL-2R binding is not affected by conjugation of NL-2/15 to the TRAP-rings and its modification with SpyTag (FIG. 20a). Treatment of cells with empty TRAP-cages did not result in any signal transduction before or after UV irradiation of the samples (FIG. 20b).

The production of SEAP was prominent after the treatment with TRAP-cage filled with NL-2 after UV irradiation (FIG. 20b). The sample without UV light treatment was also capable of signal transduction through IL-2R but on much lower level.

### Protein Production

Patchwork structure composed of TRAP variant containing K35C mutation, N-terminal His6-SUMO and SpyCatcher at either the downstream of the SUMO or between the residue 47 and 48 with TRAP-K35C (or TRAP-K35C,R64S,d73K,d74K for NL-2 encpsulation) were produced using the protocol essentially the same as the one for mCherry fusion. Tetracycline (10 or 30 ng/mL) and ITPG (0.2 mM) were used for induction of protein expression. After cell lysis by sonication, the fusion protein was purified from soluble fraction using Ni-NTA affinity chromatography. Then, His6-SUMO unit was cleaved from full-length fusion by treatment with SUMO protease 1 (25 units/mg of total protein) at 4° C. overnight, followed by treatment with Ni-NTA agarose resin to remove unreacted species and the his-tagged protease. The desired patchwork assemblies were further purified by size-exclusion chromatography. The fidelity of proteins as well as number of SpyCatcher per 11 mer TRAP ring was estimated using band intensity ratio in SDS-PAGE analysis.

N-terminally His6 and SpyTagged GFP and Neoleukin-2/15, referred to as H-SpyT-GFP or H-SpyT-NL-2, were produced using E. coli BL21(DE3) strain that were transformed with pET28_H-SpyT-GFP or pET28_H-SpyT-NL-2, a pET28-based plasmid with a ColE origin of replication, Kanamycin-resistance gene, the lac repressor, and H-SpyT-GFP or H-SpyT-NL-2 under control of T7 promoter and lac operon system. Protein was expressed using 0.2 mM IPTG at 25° C. for 20 hours, and purified using Ni-NTA affinity chromatography and size-exclusion chromatography.

Cage assembly and characterization: Patchwork TRAP ring containing SpyCatcher (400 μM respect to TRAP monomer) were mixed with TPPMS-Au(I)-CI (200 μM) in 50 mM sodium phosphate buffer (pH 8.0) containing 600 mM NaCl (2M NaCl for the one containing TRAP-K35C,R64S,d73K,d74K) and kept at room temperature overnight. Assembled cages were then isolated using size-exclusion chromatography. For the photo-openable cage, the Au(I)-mediated cage (200 μM respect to TRAP monomer) was added with 1,2-bromomethyl-3-nitrobenzene (300 μM, 3 euiv.) in DMF (final 5%) and stirred at room temperature for 1 hour. β-mercaptoethanol (4 μL) was then added to the reaction and further stirred at room temperature for 30 minutes to quench the unreacted benzylbromide and to remove Au(I). These small molecular reactants were removed by ultrafiltration using an Amicon ultra-4 centrifugal unit (30,000 molecular weight cuttoff), and the resulted cages were used for encapsulation without further purification. The number of SpyCatcher per cage was estimated using band intensity ratio in SDS-PAGE analysis. The morphology of the isolated cage was examined using negative stain TEM with the protocol described above.

Guest loading (small scale): Patchwork TRAP rings containing SpyCatcher (20 μM, respect to SpyCatcher) were mixed with H-SpyT-GFP (0-20 μM) in PBS and kept at room temperature overnight. The reaction mixtures were subsequently analyzed by SDS-PAGE and native-PAGE. For the native-PAGE analysis, the bands were visualized using both Instant Blue staining and fluorescence using a blue light excitation and an emission filter (530/28 nm) on a Biorad ChemiDoc MP imager.

Guest loading (large scale): Patchwork TRAP rings containing SpyCatcher (20 μM, respect to SpyCatcher) were mixed with H-SpyT-GFP (20 μM) or H-SpyT-NL-2 (40 μM) in PBS and kept at room temperature overnight. The cages were then isolated by size-exclusion chromatography using a Superose 6 increase 10/300 column, followed by TEM and spectroscopic analysis. TEM imaging was performed as described above. The number of the guest per cage was estimated by absorbance measured on a UV-1900 UV-Vis Spectrophotometer (Shimadzu) using extinction coefficients: ϵ_{GFP 488}=52,700 M⁻¹ cm⁻¹, ϵ_{GFP 280}=26,850 M⁻¹, ϵ_{TRAP 280}=8250 M⁻¹ cm⁻¹ (httpliexpasy.orgitoolstprotparam.html).

TABLE 3
Plasmid information and amino acid sequences

| Sequence | | | | |
|---|---|---|---|---|
| ID | Plasmid name | Plasmid | Gene | Amino acid sequence |
| SEQ ID | pACTet_SpyC- | pACYC | His6- | MHHHHHHGSSMASMKDHLIHNHH |
| NO: 12 | TRAP-K35C | | SUMO- | KHEHAHAEHLGSDSEVNQEAKPEV |
| | | | SpyCatcher- | KPEVKPETHINLKVSDGSSEIFFKIK |
| | | | TRAP- | KTTPLRRLMEAFAKRQGKEMDSLR |
| | | | K35C | FLYDGIRIQADQTPEDLDMEDNDIIE |
| | | | | AHREQIGGSDSATHIKFSKRDEDGK |
| | | | | ELAGATMELRDSSGKTISTWISDGQ |
| | | | | VKDFYLYPGKYTFVETAAPDGYEVA |
| | | | | TAITFTVNEQGQVTVNGKATKGDAH |
| | | | | IGSSYTNSDFVVIKALEDGVNVIGLTR |
| | | | | GADTRFHHSECLDKGEVLIAQFTEH |
| | | | | TSAIKVRGKAYIQTRHGVIESEGKK* |
| SEQ ID | pACTet_TRAP- | pACYC | His6- | MHHHHHHGSSMASMKDHLIHNHHK |
| NO: 13 | loopSpyC | | SUMO- | HEHAHAEHLGSDSEVNQEAKPEVKP |
| | | | TRAP-K35C | EVKPETHINLKVSDGSSEIFFKIKKTTP |
| | | | (loop | LRRLMEAFAKRQGKEMDSLRFLYDG |
| | | | SpyCatcher) | IRIQADQTPEDLDMEDNDIIEAHREQI |
| | | | | GGSGSGGSSYTNSDFVVIKALEDGV |
| | | | | NVIGLTRGADTRFHHSECLDKGEVLI |
| | | | | AQFTGSSDSATHIKFSKRDEDGKEL |
| | | | | AGATMELRDSSGKTISTWISDGQVK |
| | | | | DFYLYPGKYTFVETAAPDGYEVATAI |
| | | | | TFTVNEQGQVTVNGKATKGDAHIPG |
| | | | | TEHTSAIKVRGKAYIQTRHGVIESE |
| | | | | GKK* |
| SEQ ID | pET21_SpyT- | pET21 | SpyTag- | MAHIVMVDAYKPTKQGSGGSGSSYT |
| NO: 14 | TRAP-H | | TRAP- | NSDFVVIKALEDGVNVIGLTRGADTR |
| | | | K35C-srt- | FHHSECLDKGEVLIAQFTEHTSAIKVR |
| | | | His6 | GKAYIQTRHGVIESEGKKGTGGSLPS |
| | | | | TGGAPVEHHHHHH* |
| SEQ ID | pET28_H- | pET28 | His6- | MGSSHHHHHHGGSAHIVMVDAYKPT |
| NO: 15 | SpyT-GFP | | SpyTag- | KGSGTASKGEELFTGVVPILVELDGD |
| | | | GFP | VNGHKFSVRGEGEGDATNGKLTLKF |
| | | | | ICTTGKLPVPWPTLVTTLTYGVQCFS |
| | | | | RYPDHMKRHDFFKSAMPEGYVQERT |
| | | | | ISFKDDGTYKTRAEVKFEGDTLVNRIE |
| | | | | LKGIDFKEDGNILGHKLEYNFNSHNVY |
| | | | | ITADKQKNGIKANFKIRHNVEDGSVQL |
| | | | | ADHYQQNTPIGDGPVLLPDNHYLSTQ |
| | | | | SKLSKDPNEKRDHMVLLEFVTAAGIT |
| | | | | HGMDELYK* |
| SEQ ID | pET28_H- | pET28 | His6- | MGSSHHHHHHGGSAHIVMVDAYK |
| NO: 16 | SpyT-NL-2 | | SpyTag-NL- | PTKGSGTPKKKIQLHAEHALYDALM |
| | | | 2 | ILNIVKTNSPPAEEKLEDYAFNFELIL |
| | | | | EEIARLFESGDQKDEAEKAKRMKE |
| | | | | WMKRIKTTASEDEQEEMANAIITILQ |
| | | | | SWIFS* |
| SEQ ID | pET21_TRAP- | pET21 | TRAP | MYTNSDFVVIKALEDGVNVIGLTRG |
| NO: 17 | K35C-R64S- | | mutant | ADTRFHHSECLDKGEVLIAQFTEHTS |
| | dK73K74 | | K35C, | AIKVRGKAYIQTSHGVIESEG* |
| | | | R64S, | |
| | | | dK73, | |
| | | | dK74K | |

### HEK-Blue-IL-2 Reporter Cells Assay

HEK-Blue-IL-2 cells were cultured in DMEM-high glucose medium with 10% FBS, 100 U/mL Penicilin, 100 ug/mL Streptomycin and 50 ug/mL Normocin. After passage 2 cells were also supplemented with HEK-BIueTM CLR Selection and Puromycin to guarantee persistent transgene expression in cells. Prior to seeding CLR selection medium was exchanged to DMEM-high glucose medium with 10% FBS, 100 U/mL Penicilin, 100 ug/mL Streptomycin (P/S) and 50 ug/mL Normocin. Cells were detached from a surface of a culture bottle (VWR) by stream, centrifuged 70×g for 8 min and resuspended in 2 ml fresh medium without Normocin addition. To assess their number, 10 μl of cells suspension was transferred on a counting plate (BioRad) and and placed in TC20 Automated Cell Counter (BioRad). Cells were seeded on the 96-well culture plates (VWR) in the 180 ul culture medium in the 1×10⁴ density and incubated for 20 hours in 37° C. and 5% CO₂.

Tested proteins were prepared as 10× stock dilutions in DMEM-high glucose with 10% FBS. The next day HEK-Blue-IL-2 cells were stimulated by the addition of 20 μl of proteins in the various concentrations and incubated for 24 hours in 37° C. and 5% CO₂. Quanti-BIue™ solution was prepared by the 100× dilution of QB-buffer and QB-Reagent in sterile H₂O and incubated with gentle shaking for 10 min protected from light. 180 ul of Quanti-BlueTmsolution was transferred to each well of the fresh 96-well plate and added with 20 μl of HEK-Blue-IL-2 cells supernatant. The plate was incubated for 1 hour in 37° C. Secreted embryonic alkaline phosphatase (SEAP) activity was assessed by the absorbance measurement at 630 nm.

### REFERENCES

1 Malay, A. D. et al. An ultra-stable gold-coordinated protein cage displaying reversible assembly. Nature 569, 438-442 (2019).
2 Butterfield, G. L. et al. Evolution of a designed protein assembly encapsulating its own RNA genome. Nature 552, 415-420 (2017).
3 Edwardson, T. G., Mori, T. & Hilvert, D. Rational Engineering of a Designed Protein Cage for siRNA Delivery. J. Am. Chem. Soc. (2018).
4 Azuma, Y., Zschoche, R., Tinzl, M. & Hilvert, D. Quantitative packaging of active enzymes into a protein cage. Angew. Chem. Int. Ed. 55, 1531-1534 (2016).
5 Dashti, N. H., Abidin, R. S. & Sainsbury, F. Programmable in vitro coencapsidation of guest proteins for intracellular delivery by virus-like particles. ACS nano 12, 4615-4623 (2018).
6 Wörsdörfer, B., Pianowski, Z. & Hilvert, D. Efficient in vitro encapsulation of protein cargo by an engineered protein container. Journal of the American Chemical Society 134, 909-911 (2012).
7 Ho, A., Schwarze, S. R., Mermelstein, S. J., Waksman, G. & Dowdy, S. F. Synthetic protein transduction domains: enhanced transduction potential in vitro and in vivo. Cancer research 61, 474-477 (2001).
8 Berry, C. C. Intracellular delivery of nanoparticles via the HIV-1 tat protein. Nanomedicine 3, 357 - 365 (2008).
9 Rampersad, S. N. Multiple applications of Alamar Blue as an indicator of metabolic function and cellular health in cell viability bioassays. Sensors 12, 12347-12360 (2012).

1. An artificial TRAP-cage comprising a selected number of TRAP rings and encapsulated therein at least one guest cargo.

2. The cage according to claim 1, wherein the guest cargo is selected from the group comprising a protein, an enzyme, an antigen, an antibody. a protein macromolecule a lipid, a peptide, a nucleic acid, a small molecular cargo, a peptide nucleic acid, a carbon- based structure, a metal, a toxin or a nanoparticle.

3. The cage according to claim 2, wherein the nucleic acid is selected from the group comprising DNA, RNA, mRNA, siRNA, tRNA and micro-RNA.

4. The cage according to claim 2, wherein the enzyme is an enzyme associated with an over-expression in a metabolic disorder or disease or an underexpression in a metabolic disorder or disease.

5. The cage according to claim 4, wherein the enzyme is selected from the group comprising hydrogenase, dehydrogenase, lipase, lyase, ligase, protease, transferase, reductase, recombinase and nuclease acid modification enzyme.

6. The cage according to claim 2, wherein the therapeutic agent is selected from the group comprising a cancer therapeutic, an anti-infection therapeutic, a vascular disease therapeutic, an immune therapeutic, senolytic and a neurological therapeutic.

7. The cage according to claim 2 wherein the metal is selected from the group comprising iron, zinc, platinum, copper, sodium, cadmium, lanthanide, gadolinium, technetium, calcium, potassium, chromium, magnesium, molybdenum and salts or complexes thereof.

8. The cage according to claim 2 wherein the toxin is selected from the group comprising a ligand targeted toxin, a protease activated toxin, melittin and a toxin-based suicide gene therapeutic.

9. The cage according to any preceding claim, wherein the guest cargo is a protein and preferably the protein is a fluorescent protein, interleukin-2 (IL-2) or Neoleukin-2/15 (NL-2).

10. The cage according to any preceding claim wherein the cage comprises multiple guest cargos and wherein the guest cargoes are the same or different from one another, and are any combination of the cargos from claims 2 to 9.

11. The cage according to any preceding claim, further including at least one external decoration.

12. The cage according to claim 11, wherein at least one of the external decorations comprises a cell penetrating agent to promote intracellular delivery of the cage containing an internal guest cargo.

13. The cage according to claim 12, wherein the cell penetrating agent is PTD4.

14. The cage according to any preceding claim, wherein the number of TRAP rings in the TRAP-cage is between 6 to 60.

15. The cage according to claim 14, wherein the number of TRAP rings in the TRAP-cage is 12, 20 or 24, preferably 24.

16. The cage according to any preceding claim, wherein the interior surface of the TRAP-cage lumen is supercharged and the TRAP-cage protein comprises a E48Q or a E48K mutation.

17. The cage according to any preceding claim, wherein the artificial TRAP-cage protein is modified to comprise any one or more of the following mutations selected from the group comprising, comprising K35C, K35H, R64S, E48Q, E48K, K35C/R64S, K35H/R64S, S33C, S33H, S33C/R64S, S33H/R64S, S33C/K35H S33H/K35H, S33C/K35C, S33H/K35C, K35C/E48Q, K35C/E48K, K35H/E48Q, K35H/E48K, S33C/E48Q, S33C/E48K, S33C/E48Q and S33C/E48K.

18. The TRAP-cage according to any preceding claim, wherein opening of the cage is programmable.

19. The TRAP-cage according to claim 18, wherein the programmable opening of the cage is dependent on selection of a molecular or atomic cross-linker which hold the TRAP-rings in place in the TRAP-cage.

20. The TRAP-cage according to claim 19, wherein the cross-linker is either (i) a reduction responsive/sensitive linker, whereby the cage opens under reduction conditions; or (ii) a photo-activatable linker whereby the cage opens upon exposure to light.

21. Use of the artificial TRAP-cage according to any preceding claim as a delivery vehicle for intracellular delivery of its internal guest cargo.

22. Use of the artificial TRAP-cage according to any one of claims 1 to 20 as a vaccine.

23. Use of the artificial TRAP-cage according to any one of claims 1 to 20 for the treatment of an illness or disease condition selected from the group comprising cancer, vascular disease, cardiovascular disease, diabetes, infection, auto-immune condition, neurodegenerative disease, cellular senescence disease, arthritis and respiratory disease.

24. A method of making an artificial TRAP-cage with an encapsulated guest cargo, the method comprising:
(i) obtaining TRAP ring units by expression of the TRAP ring units in a suitable expression system and purification of the said units from the expression system;
(ii) conjugation of the TRAP ring units via at least one free thiol linkage with a cross-linker;
(iii) modification of the TRAP ring units to provide a suitable interior surface environment for capturing a guest cargo;
(iv) formation of the TRAP-cage by self-assembly to provide a cage lumen wherein the guest cargo is encapsulated; and
(v) purification and isolation of the TRAP-cages encapsulating the guest cargo.

25. The method of claim 24 wherein the modification of step (iii) is selected from the group comprising:
(i) super charging the interior surface of the TRAP-cage lumen;
(ii) genetic fusion of the guest cargo to an interior surface of the TRAP-cage lumen;
(iii) SpyCatcher/SpyTag conjugation of the guest cargo to an interior surface of the TRAP-cage lumen; and
(iv) via covalent bond formation in both chemical and enzymatic methods.

26. The method of claim 24 or 25 wherein step (ii) first comprises conjugation of the TRAP ring units via at least one metal cross-linker, preferably an atomic metal cross-linker, then replacing the metal cross-linker with a molecular cross-linker.

27. The method according to any one of claims 24 to 26, wherein the super charging of step (i) of the interior surface provides either a net positive or net negative charge on the interior surface of the cage lumen.

28. The method according to any one of claims 24 to 27, wherein the artificial TRAP-cage protein is modified to comprise any one or more of the following mutations selected from the group comprising, comprising K35C, K35H, R64S, E48Q, E48K, K35C/R64S, K35H/R64S, S33C, S33H, S33C/R64S, S33H/R64S, S33C/K35H S33H/K35H, S33C/K35C, S33H/K35C, K35C/E48Q, K35C/E48K, K35H/E48Q, K35H/E48K, S33C/E48Q, S33C/E48K, S33C/E48Q and S33C/E48K.

29. The method according to any of claim 28 wherein the cage formation step of part (iii) for TRAP^{K35C E48Q} is performed in sodium bicarbonate buffer at pH 9-11.

30. The method according to any of claim 28 wherein the cage formation step of part (iii) for TRAP^{K35C E48k} is performed in sodium bicarbonate buffer at pH 10-10.5.

31. The method according to any one of claims 24 to 30, wherein the guest cargo can be loaded either pre or post assembly of the TRAP-cage.

32. The method according to any one of claims 24 to 31, wherein the genetic fusion of the guest cargo to an interior surface of the TRAP-cage lumen of step (ii) is via N-terminus fusion of the guest cargo to an N-terminus of TRAP^{K35C} which faces into the interior surface of the lumen.

33. The method according to claim 32, wherein the SpyCatcher/SpyTag conjugation of the guest cargo to an interior surface of the TRAP-cage lumen of step (iii) wherein the SpyCatcher is introduced in a loop region of TRAP rings between residues 47 and 48, which faces to the interior when assembled into TRAP-cages and the guest cargo contains a SpyTag.

34. The method according to any one of claims 24 to 33, wherein enzymatic modification is via peptide ligase selected from the group comprising sortases, asparaginyl endoproteases, trypsin related enzymes and subtilisin-derived variants and covalent chemical bond formation may include strain promoted alkyne-azide cycloaddition and pseudopeptide bonds.

35. A TRAP cage produced by method of any one of claims 24 to 34.

36. Use of the cage according to any one of claims 1 to 20 as a medicament.

37. A method of treating a patient, comprising administering a cage according to any one of claims 1 to 20 to said patient.

38. The cage according to any one of claims 1 to 20 for use in treating a disease in a patient or as a vaccine.

39. An artificial TRAP-cage protein modified to comprise any one or more of the following mutations selected from the group comprising K35C, K35H, R64S, E48Q, E48K, K35C/R64S, K35H/R64S, S33C, S33H, S33C/R64S, S33H/R64S, S33C/K35H S33H/K35H, S33C/K35C, S33H/K35C, K35C/E48Q, K35C/E48K, K35H/E48Q, K35H/E48K, S33C/E48Q, S33C/E48K, S33C/E48Q and S33C/E48K.

40. A method of treatment of an individual in need of therapy suffering from a condition selected from the group comprising cancer, vascular disease, cardiovascular disease, diabetes, infection, auto-immune condition, neurodegenerative and neurological disease, cellular senescence disease, arthritis and respiratory disease, the method comprising administering a therapeutically effective amount of an artificial TRAP-cage bearing one or more internal guest cargo selected from the group comprising a nucleic acid, an enzyme, a therapeutic agent, a small molecule, organic or inorganic nanoparticles, a peptide, a metal, an antigen, an antibody and toxin and fragments thereof of all the foregoing that are of therapeutic value.

41. A method of vaccinating an individual in need of vaccination from a condition selected from the group comprising cancer, vascular disease, cardiovascular disease, diabetes, infection, auto-immune condition, neurodegenerative and neurological disease, cellular senescence disease, arthritis and respiratory disease, the method comprising administering a therapeutically effective amount of an artificial TRAP-cage bearing one or more internal guest cargo selected from the group comprising a nucleic acid, an enzyme, a therapeutic agent, a small molecule, organic or inorganic nanoparticles, a peptide, a metal, an antigen, an antibody and toxin and fragments thereof of all the foregoing that are of therapeutic value

42. The methods of either claim 40 or 41 wherein the TRAP-cage therapeutic is administered via intranasal inhalation or injection.
